SEQUENCE LISTING (33 sequences)

SEQ ID NO: 1 - protein, 7 aa, Artificial Sequence
  Chemically synthesized
LTAQPST

SEQ ID NO: 2 - protein, 10 aa, Artificial Sequence
  Chemically synthesized
ACTVRTSADC

SEQ ID NO: 3 - protein, 7 aa, Artificial Sequence
  Chemically synthesized
VNRSSLY

SEQ ID NO: 4 - protein, 7 aa, Artificial Sequence
  Chemically synthesized
GETRAPL

SEQ ID NO: 5 - protein, 7 aa, Artificial Sequence
  Chemically synthesized
APTAVSK

SEQ ID NO: 6 - protein, 7 aa, Artificial Sequence
  Chemically synthesized
TPHPLRL

SEQ ID NO: 7 - protein, 7 aa, Artificial Sequence
  Chemically synthesized
APKQSLE

SEQ ID NO: 8 - protein, 7 aa, Artificial Sequence
  Chemically synthesized
VSTPSTP

SEQ ID NO: 9 - protein, 7 aa, Artificial Sequence
  Chemically synthesized
GGLSSRP

SEQ ID NO: 10 - protein, 7 aa, Artificial Sequence
  Chemically synthesized
YPSPWGY

SEQ ID NO: 11 - protein, 7 aa, Artificial Sequence
  Chemically synthesized
TLNRVPN

SEQ ID NO: 12 - protein, 7 aa, Artificial Sequence
  Chemically synthesized
GVPTALP

SEQ ID NO: 13 - protein, 7 aa, Artificial Sequence
  Chemically synthesized
QLVYPAP

SEQ ID NO: 14 - protein, 7 aa, Artificial Sequence
  Chemically synthesized
SSQLSRP

SEQ ID NO: 15 - protein, 7 aa, Artificial Sequence
  Chemically synthesized
LGTSMQL

SEQ ID NO: 16 - protein, 7 aa, Artificial Sequence
  Chemically synthesized
SLGPSPG

SEQ ID NO: 17 - protein, 7 aa, Artificial Sequence
  Chemically synthesized
ISLPSPT

SEQ ID NO: 18 - protein, 7 aa, Artificial Sequence
  Chemically synthesized
MISSNSS

SEQ ID NO: 19 - protein, 7 aa, Artificial Sequence
  Chemically synthesized
YNSPTHH

SEQ ID NO: 20 - protein, 7 aa, Artificial Sequence
  Chemically synthesized
SGTHHKA

SEQ ID NO: 21 - protein, 7 aa, Artificial Sequence
  Chemically synthesized
TNTMTRA

SEQ ID NO: 22 - protein, 7 aa, Artificial Sequence
  Chemically synthesized
KPFPPMK

SEQ ID NO: 23 - protein, 7 aa, Artificial Sequence
  Chemically synthesized
ETTHLTG

SEQ ID NO: 24 - protein, 8 aa, Artificial Sequence
  Chemically synthesized
SPHDVAYD

SEQ ID NO: 25 - protein, 7 aa, Artificial Sequence
  Chemically synthesized
QLKPLEF

SEQ ID NO: 26 - protein, 7 aa, Artificial Sequence
  Chemically synthesized
LPLWEVY

SEQ ID NO: 27 - protein, 7 aa, Artificial Sequence
  Chemically synthesized
TVRTSAD

SEQ ID NO: 28 - protein, 7 aa, Artificial Sequence
  Chemically synthesized
NTGSPYE

SEQ ID NO: 29 - protein, 11 aa, Artificial Sequence
  Chemically synthesized
ISLPSPTGGGS

SEQ ID NO: 30 - protein, 11 aa, Artificial Sequence
  Chemically synthesized
SSQLSRPGGGS

SEQ ID NO: 31 - protein, 11 aa, Artificial Sequence
  Chemically synthesized
YNSPTHHGGGS

SEQ ID NO: 32 - protein, 8 aa, Artificial Sequence
  Chemically synthesized
TEASGGGC

SEQ ID NO: 33 - protein, 8 aa, Artificial Sequence
  Chemically synthesized
CASHSYFP

(19) United States
(12) Patent Application Publication
(10) Pub. No.: US 2021/0284688 A1
(43) Pub. Date: Sep. 16, 2021

(54) MUCUS-PENETRATING PEPTIDES AND SCREENING ASSAY

(71) Applicant: BOARD OF REGENTS, THE UNIVERSITY OF TEXAS SYSTEM, Austin, TX (US)

(72) Inventors: Debadyuti GHOSH, Austin, TX (US); Hugh SMYTH, Austin, TX (US)

(21) Appl. No.: 16/317,843

(22) PCT Filed: Jul. 14, 2017
(86) PCT No.: PCT/US17/42102

§ 371 Date: Jan. 15, 2019

Related U.S. Application Data
(60) Provisional application No. 62/362,781, filed on Jul. 15, 2016.

(51) Int. Cl. C07K 7/06; A61K 47/64; C12N 15/10

(52) U.S. Cl. CPC C07K 7/06; C12N 15/1037; A61K 47/64

(57) ABSTRACT

The present invention relates to a screening method to identify mucus-penetrating compounds. In certain aspects, the present invention relates to mucus-penetrating peptides and constructs comprising an agent, such as a therapeutic agent, imaging agent or diagnostic agent, conjugated to a mucus-penetrating peptide.

### CROSS REFERENCE TO RELATED APPLICATIONS

This application claims priority to U.S. Provisional Application No. 62/362,781, filed Jul. 15, 2016, which is hereby incorporated by reference herein in its entirety.

### BACKGROUND OF THE INVENTION

Mucosal surfaces of gastrointestinal, vaginal, respiratory and nasal tissues provide innate protection from pathogens and allow passage of nutrients for tissue homeostasis. However, in diseases such as cystic fibrosis, mucus is aberrantly expressed and creates a local environment to trap and protect pathogens resulting in chronic bacterial infections while concomitantly rendering drugs incapable of penetrating the physiological barriers. To improve upon drug delivery strategies, it is thus critical to enhance penetration through the mucus barrier. While recent studies have shown formulations of hydrophilic, net-neutral charge polymers can improve transport and minimize interactions with mucus (McGill and Smyth, 2010, Mol Pharm, 7: 2280-2288; Wang et al., 2008, Angew Chem Int Ed Engl, 47: 9726-9729; Lai et al., 2007, Proc Natl Acad Sci USA, 104: 1482-1487; Olmsted et al., 2001, Biophys J, 81: 1930-1937), work has been limited to a small number of formulations and subsequently, comprehensive studies of particle-mucin interactions have not been achieved.

Thus, there is a need in the art for compositions and methods for delivery of agents through the mucus barrier. The present invention satisfies this unmet need.

### SUMMARY OF THE INVENTION

In one aspect, the present invention provides a composition comprising one or more mucus-penetrating peptides. In one embodiment, the one or more mucus-penetrating peptides comprises a peptide comprising an amino acid sequence selected from the group consisting of: an amino acid sequence selected from SEQ ID NOs: 1-28, an amino acid sequence having at least 70% homology to any one of SEQ ID NOs: 1-28, and a fragment of an amino acid sequence selected from SEQ ID NOs: 1-28.

In one embodiment, the composition further comprises at least one agent selected from the group consisting of: a therapeutic agent, prophylactic agent, diagnostic agent, imaging agent, contrast agent, microparticle, and nanoparticle. In some embodiments, the agent is at least one selected from the group consisting of a peptide, nucleic acid molecule, small molecule drug, organic compound, and inorganic compound. In one embodiment, the composition comprises a fusion construct comprising one or more mucus-penetrating peptides conjugated to the at least one agent.

In one aspect, the present invention provides a composition comprising an isolated nucleic acid molecule encoding a mucus-penetrating peptide. In one embodiment, the isolated nucleic acid molecule encodes a mucus-penetrating peptide comprising an amino acid sequence selected from the group consisting of: an amino acid sequence selected from SEQ ID NOs: 1-28, an amino acid sequence having at least 70% homology to any one of SEQ ID NOs: 1-28, and a fragment of an amino acid sequence selected from SEQ ID NOs: 1-28.

In one aspect, the present invention provides a method of delivering an agent across a mucosal barrier comprising administering to the mucosal barrier a composition comprising the agent and one or more mucus-penetrating peptides. In one embodiment, the one or more mucus-penetrating peptides comprises a peptide comprising an amino acid sequence selected from the group consisting of: an amino acid sequence selected from SEQ ID NOs: 1-28, an amino acid sequence having at least 70% homology to any one of SEQ ID NOs: 1-28, and a fragment of an amino acid sequence selected from SEQ ID NOs: 1-28.

In one embodiment, the agent is at least one selected from the group consisting of a therapeutic agent, prophylactic agent, diagnostic agent, imaging agent, contrast agent, microparticle, and nanoparticle. In one embodiment, the composition comprises a fusion construct comprising the one or more mucus-penetrating peptides conjugated to the agent.

In one aspect, the present invention provides a method of treating a disease or disorder in a subject by delivery of a therapeutic or prophylactic agent through a mucosal barrier in a subject, the method comprising administering to the subject a composition comprising the therapeutic or prophylactic agent and one or more mucus-penetrating peptides. In one embodiment, the one or more mucus-penetrating peptides comprises a peptide comprising an amino acid sequence selected from the group consisting of: an amino acid sequence selected from SEQ ID NOs: 1-28, an amino acid sequence having at least 70% homology to any one of SEQ ID NOs: 1-28, and a fragment of an amino acid sequence selected from SEQ ID NOs: 1-28.

In one embodiment, the composition comprises a fusion construct comprising the one or more mucus-penetrating peptides conjugated to the therapeutic or prophylactic agent.

In one aspect, the present invention provides a method of screening for a compound capable of penetrating a mucosal barrier. In on embodiment, the method comprises providing a container comprising a first chamber, a second chamber, and a permeable membrane separating the first chamber and second chamber, wherein the first chamber comprises mucus or mucus-like substance; administering one or more test compounds to the first chamber; and collecting the contents of the second chamber at a time point following the administration of the one or more test compounds.

In one embodiment, the method further comprises one or more rounds of re-administering the collected contents of the second chamber into the first chamber and collecting the contents of the second chamber. In one embodiment, the method comprises a phage library-based assay, comprising administering a plurality of peptide-expressing phage to the first chamber, and collecting the phage in the second chamber.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts phage peptides with their frequency and functional side chain properties identified in screening using 20% w/v mucin.

FIG. 2 depicts a diagram illustrating the mucin barrier to lung epithelial cells that traps pathogens and minimizes drug penetration. Adapted from Barr and Auro, Proceedings of the National Academy of Sciences, 110, 25 Jun. 2013.

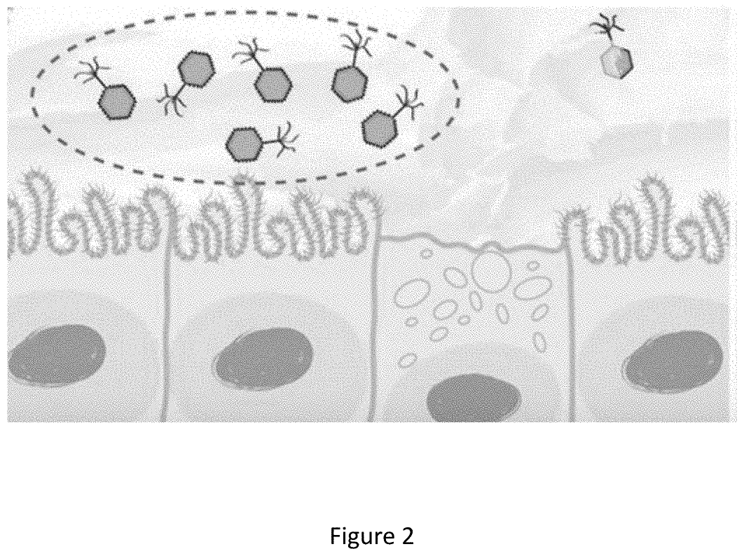

FIG. 3 depicts a 3-D rendering of the M13 bacteriophage and its different capsid proteins.

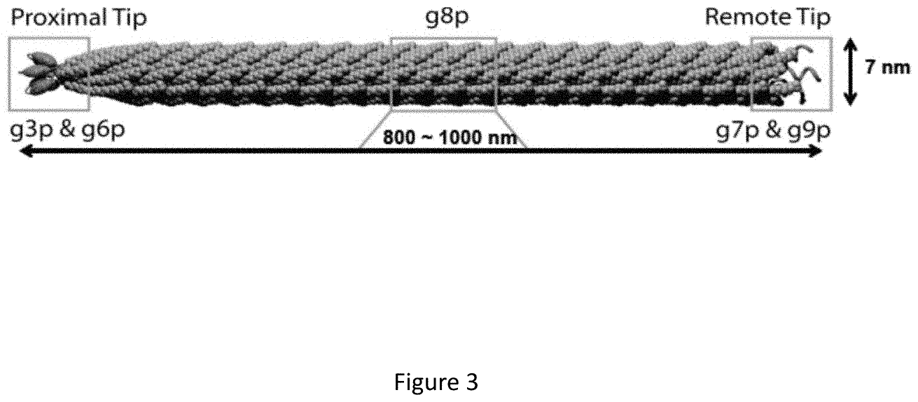

FIG. 4 depicts a plasmid map of M13KE revealing the pIII gene used for phage display of random peptides and lacZ gene used in blue-white screening. Random oligonucleotides encode 7-mer peptide library that is engineered into the N-terminus of pIII. The phage library can achieve up to 2×10⁹ in diversity. [N=A,C,T,G, K=T,C]

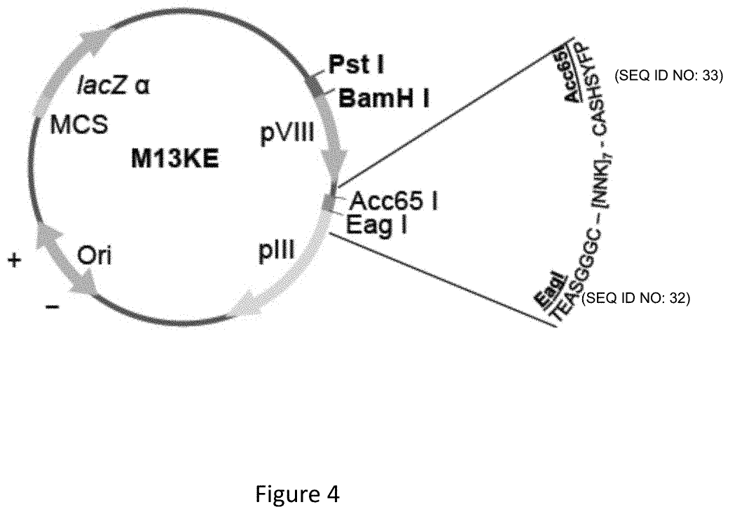

FIG. 5, comprising FIG. 5A and FIG. 5B depicts experimental results demonstrating a preliminary library screen. FIG. 5A depicts titering of 2 hour time point from preliminary library screen using blue-white screening technique. Serial 10-fold dilutions of phage were incubated with early-log culture of E. coli, plated on agar and overlaid with top agar. FIG. 5B depicts a schematic demonstrating that plaques indicating areas of phage infected bacteria are blue due to the interaction of X-gal with expressed β-galactosidase from phage infected bacteria (adopted from Oxford Genetics).

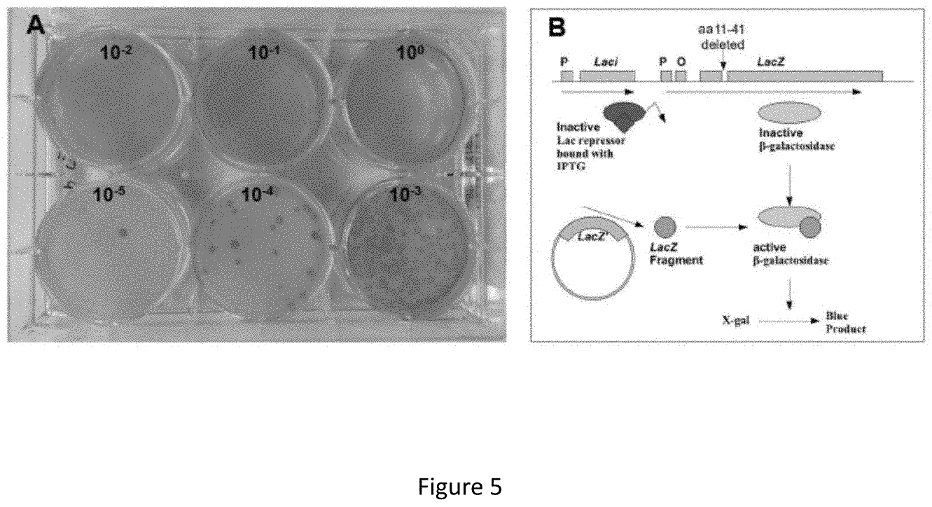

FIG. 6 depicts an overview of the phage penetration assay for mucin. (A) Mucin is incubated in the donating reservoir. (B) 10¹⁰ phage of the phage library is added to the donating reservoir. Phage that penetrate through mucin and semipermeable membrane are collected. (C) Eluted phage is quantified through titering and is amplified by E. coli for next round of screening. (D) Amplified phage is quantified so that equivalent phage is added for each round. (E) Top down view and (F) side view of the a transwell with mucin layer for screens against mucin.

FIG. 7, comprising FIG. 7A and FIG. 7B, depicts the quantification and validation of transported phage through hyperconcentrated mucin. FIG. 7A depicts titering results of phage eluate at 1 hour timepoint against a mucin layer. FIG. 7B depicts a comparison of titering results of phage eluate at 1 hour timepoint between positive clones from round 3 and the wild-type negative controls. *=p<0.05.

FIG. 8 depicts the quantification of selected phage through complex mucin. Titering results of the phage eluate at 1 hour timepoint against a complex mucin formulation containing lipids, protein cell debris, and salts. An enrichment in the number of phages that are transported across the mucus layer can be seen markedly in round 4.

FIG. 9 depicts the enhanced diffusivities of selected mucin-penetrating M13 phage (left). Diffusivities of selected phage S1 (left) and negative control in 8% mucin are depicted (center and right). Results show the diffusivities of selected phage B and C and negative control in complex mucin.

FIG. 10 depicts identified sequences and their physiochemical properties. Peptide sequences from round 4 eluates from complex mucin screens.

FIG. 11 depicts the hydrophilicity of mucin-penetrating clones. Kyte-Doolittle hydropathy plot of sequences 13 and 14 from FIG. 10. X-axis is amino acid position and y-axis denotes hydropathy score assigned to amino acid. Negative score represents hydrophilic amino acids and positive scores represent hydrophobic amino acids. From the collected sequences, the average hydrophobicity score at each amino acid position is calculated. Adopted from Kyte and Doolittle, Journal of Molecular Biology. 157, 1982.

FIG. 12 depicts physicochemical properties of three selected isolated sequences, SEQ ID NO: 17, SEQ ID NO: 14, and SEQ ID NO: 19, where each sequence in these studies further contained the flexible linker GGGS, as this linker is engineered into the p3 library for N-terminal display of peptides.

FIG. 13 depicts results from example experiments, demonstrating diffusion results for the three selected M13 clones, C/Co versus time (seconds). M13KE served as a control. Here, C is the concentration of phage that transported across mucin layer into the receiving chamber, and Co is the initial concentration of phage.

FIG. 14 depicts results from example experiments, demonstrating dynamic light scattering (DLS) measurements of the four phage-presenting peptides in PBS. Diffusion coefficients are displayed in cm²/s for each sample for n=3 (for each phage clone, DLS measurements were taken in triplicate).

FIG. 15 depicts results from example experiments, demonstrating the effective diffusivity of clones in complex mucin (CM) compared to the effective diffusivity of clones in PBS. Shown are phage diffusivities in PBS, CM, and the ratio of diffusivity in CM to PBS for the four phage-presenting peptides.

FIG. 16 depicts results from example experiments, demonstrating effective diffusion coefficients (cm²/sec) of fluorescein samples through PBS (black bars) and complex mucin (CM, grey bars). AK10 and Dextran 40 kDa are controls.

FIG. 17 depicts results from example experiments, demonstrating effective diffusion coefficients (cm²/sec) of fluorescein samples through PBS (first column) and complex mucin (CM, second column), and the diffusion coefficient ratio (MC/PBS, third column). AK10 and Dextran 40 kDa are controls.

### DETAILED DESCRIPTION

In one aspect, the present invention relates to a method of identifying peptides able to penetrate a mucosal barrier. In one embodiment, the present invention relates to a composition comprising a mucus-penetrating peptide.

In certain aspects, the peptides described herein serve as permeation enhancers to improve transport of an agent (e.g. therapeutic agent, prophylactic agent, imaging agent, diagnostic agent) through barriers. For example, in certain instances, the peptides described herein cause openings or permeation of the barriers to permit delivery of an agent. In one embodiment, the composition is a fusion construct comprising a mucus-penetrating peptide described herein conjugated to an agent, wherein the mucus-penetrating peptide allows for the transport of the fusion construct through a mucosal barrier. However, in certain instances, the peptides described herein can facilitate transport of an agent without being physically conjugated to the agent. For example, in certain embodiments, co-administration of a peptide described herein and an agent facilitates transport of the agent across a barrier.

In one embodiment, the present invention relates to a method of delivering a composition through a mucosal barrier, contacting a mucosal surface or mucosal barrier with a composition comprising a mucus-penetrating peptide.

In certain embodiments, the mucus penetrating peptides could be used for delivery of a therapeutic agent, prophylactic agent, diagnostic agent, or imaging agent to treat, prevent, or detect various types of diseases or disorders of the mucosal epithelia, including, but not limited to, HIV, chronic obstruction pulmonary disease (COPD), diseases of the gastrointestinal tract. Further, the peptides described herein could be applied towards any application of oral drug delivery. For example, in certain embodiments the peptides described herein can be used to improve oral delivery of an agent, where the peptide aids in the agent crossing the mucosal epithelia of the gastrointestinal tract to get into the bloodstream.

### Definitions

As used herein, each of the following terms has the meaning associated with it in this section.

The articles “a” and “an” are used herein to refer to one or to more than one (i.e., to at least one) of the grammatical objects of the article. By way of example, “an element” means one element or more than one element.

“About” as used herein when referring to a measurable value such as an amount, a temporal duration, and the like, is meant to encompass variations of ±20% or ±10%, or ±5%, or ±1%, or ±0.1% from the specified value; as such variations are appropriate to perform the disclosed methods.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double-stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, oral cancer and the like.

“Cystic Fibrosis” (CF), as used herein, refers to a disease characterized by enhanced mucus accumulation in the lung, which can be accompanied by microbial infections and ultimately causes death. In some instances CF is an inherited genetic disease resulting from one or more mutations in the gene encoding the cystic fibrosis transmembrane conductance regulator (CFTR). Mutations in CFTR endogenously expressed in respiratory epithelia lead to reduced apical anion secretion causing an imbalance in ion and fluid transport. In addition to respiratory disease, some CF patients suffer from gastrointestinal problems and pancreatic insufficiency that, if left untreated, result in death.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. As used herein, the term “fragment,” as applied to a protein or peptide, refers to a subsequence of a larger protein or peptide.

The term “functionally equivalent” as used herein refers to a polypeptide according to the invention that preferably retains at least one biological function or activity of the specific amino acid sequence of either the first or second peptide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Instructional material,” as that term is used herein, includes a publication, a recording, a diagram, or any other medium of expression which can be used to communicate the usefulness of the nucleic acid, polypeptide, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” when used herein refers to a detectable compound or composition that is conjugated directly or indirectly to a molecule to generate a “labeled” molecule. The label may be detectable by itself (e.g. radioisotope labels or fluorescent labels) or, in the case of an enzymatic label, may catalyze chemical alteration of a substrate compound or composition that is detectable (e.g., avidin-biotin).

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Individual miRNAs have been identified and sequenced in different organisms, and they have been given names. Names of miRNAs and their sequences are provided herein. Additionally, other miRNAs are known to those of skill in the art and can be readily implemented in embodiments of the invention. The methods and compositions should not be limited to miRNAs identified in the application, as they are provided as examples, not necessarily as limitations of the invention.

By the term “modulating,” as used herein, is meant mediating a detectable increase or decrease in the level of a mRNA, polypeptide, or a response in a subject, or a cell or tissue of a subject, as compared with the level of a mRNA, polypeptide or a response in the subject, or a cell or tissue of the subject, in the absence of a treatment or compound, and/or compared with the level of a mRNA, polypeptide, or a response in an otherwise identical, but untreated subject, or cell or tissue of the subject. The term encompasses perturbing and/or affecting a native signal or response thereby mediating a beneficial therapeutic response in a subject.

“Mucus,” as used herein, refers to a viscoelastic natural substance containing primarily mucin glycoproteins and other materials, which protects epithelial surface of various organs/tissues, including respiratory, nasal, cervicovaginal, gastrointestinal, rectal, visual and auditory systems.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982), which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including, but not limited to deletion, insertion, substitution of one or more nucleotides, or fusion to other polynucleotide sequences.

In another embodiment, the terms “ribonucleotide,” “oligoribonucleotide,” and “polyribonucleotide” refers to a string of at least 2 base-sugar-phosphate combinations. The term includes, in another embodiment, compounds comprising nucleotides in which the sugar moiety is ribose. In another embodiment, the term includes both RNA and RNA derivates in which the backbone is modified. “Nucleotides” refers, in another embodiment, to the monomeric units of nucleic acid polymers. RNA may be, in an other embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al., Genes & Devel 16: 2491-96 and references cited therein). In addition, these forms of RNA may be single, double, triple, or quadruple stranded. The term also includes, in another embodiment, artificial nucleic acids that may contain other types of backbones, but the same bases. In another embodiment, the artificial nucleic acid is a PNA (peptide nucleic acid). PNA contain peptide backbones and nucleotide bases and are able to bind, in another embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al. Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross-reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention relates to a method of identifying peptides able to penetrate a mucosal barrier. For example, in one embodiment, the present invention relates to a screening assay of testing a population of candidate peptides to identify which of the candidate peptides displays the ability to penetrate, or pass through, a mucosal barrier or mucus-like barrier.

In certain aspects, the screening assay makes use of phage display, which allows for the screening of an unprecedented number of peptide sequences (i.e. 10⁸-10⁹) that reveal important features of mucus penetrating chemistries in mucus and mucus-like barriers, which cannot be achieved using current chemical syntheses. In certain aspects, the screening assay utilizes an iterative selection strategy, which allows for the selection for peptides that are muco-inert and mucus-penetrating irrespective of the heterogeneity of mucus composition. A phage display-based screening assay allows for the first time a biologically-based discovery assay intentionally or explicitly applied to extracellular barriers.

In one embodiment, the present invention relates to a composition comprising a mucus-penetrating peptide. In certain embodiments, the mucus-penetrating peptide is hydrophilic, or is enriched in hydrophilic amino acids. The mucus-penetrating peptide formulations circumvent the potential limitations presented by state-of-the-art PEG formulations. Further, the mucus-penetrating peptide formulations are of small size, easily amenable for bioconjugation, and offer greater chemical complexity (i.e. more diverse physicochemical properties) to potentially achieve better penetration and transport and negligible immune response.

The identified peptides can be used as permeation enhancers to facilitate the transport of an agent (e.g., therapeutic, prophylactic, diagnostic, or imaging agents) through a barrier. In certain instances, the peptides can overcome the physical and transport barriers presented by the mucus layer in various diseases. In certain instances, the peptides can cause openings or permeation of the barriers to permit delivery of the agent.

In one embodiment, the composition comprises a peptide described herein and an agent. In some embodiments, the peptide is conjugated to the agent. In some embodiments, the peptide is not conjugated to the agent. For example, in certain instances, the peptide can facilitate transport of the agent through a barrier without being physically coupled to the agent.

In one embodiment, the composition is a fusion construct comprising the mucus-penetrating peptide. For example, the fusion construct may comprise an agent, for example a therapeutic agent, prophylactic agent, diagnostic agent, imaging agent, contrast agent, microparticle, nanoparticle, or the like, fused to or conjugated to the mucus-penetrating peptide. In one embodiment, the composition comprises an agent coated with one or more mucus-penetrating peptides. In one embodiment, the composition comprises an agent conjugated to one or more mucus-penetrating peptides.

In one embodiment, the present invention relates to a method of delivering a composition through a mucosal barrier, comprising contacting a mucosal surface or mucosal barrier with a composition comprising a mucus-penetrating peptide. For example, in one embodiment, the method comprises a therapeutic or prophylactic method comprising contacting a mucosal surface or mucosal barrier with a fusion construct comprising a therapeutic or prophylactic agent fused to a mucus-penetrating peptide.

Screening Method

In one aspect, the present invention relates to a method of identifying a compound having the ability to penetrate or pass through a mucosal barrier, mucosal surface, or mucosal membrane.

In one embodiment, the method comprises administering a test compound to a container having a first chamber, a second chamber, and a permeable membrane separating the first and second chamber. In one embodiment, the first chamber comprises a mucosal barrier thereby restricting access of the test compound to the second chamber. For example, in certain embodiment, the first chamber comprises mucin. In one embodiment, the first chamber comprises mucin in the range of about 0.1% to about 50%. In one embodiment, the first chamber comprises mucin in the range of about 5% to about 20%. In one embodiment, the first chamber comprises complex mucin comprising one or more of mucin, protein debris, lipids, and salts. In one embodiment, the method comprises administering the test compound to the first chamber, and collecting the contents of the second chamber at one or more time points following the administrating of the test compound. In one embodiment, the method comprises detecting the presence or amount of the test compound in the collected contents of the second chamber.

In one embodiment, the method comprises administering a plurality of test compounds to the first chamber, and detecting which of the plurality of test compounds are present in the contents of the second chamber.

In certain embodiments, the method comprises repeated screening of test compounds that have been collected in the second chamber. For example, in certain embodiments, the collected contents of the second chamber are administered to the first chamber, and the contents of the second chamber are collected again at one or more time points. In certain instances, the repeated screening enriches the mucus-penetrating compounds.

The test compounds may be any suitable type of compound, including, but not limited to peptides, nucleic acid molecules, small molecules, organic compounds, and the like. In certain embodiments, the test compounds comprise a phage or virus. In certain embodiments, the phage or virus expresses a surface peptide, wherein the peptide directs transport of the phage or virus through the mucosal barrier. Any suitable phage or virus may be used, including but not limited to M13 bacteriophage, T7 bacteriophage, cowpea mosaic virus, MS2 bacteriophage, P22 bacteriophage, Q beta bacteriophage, and tobacco mosaic virus, adeno-associated virus, and adenovirus.

In one embodiment, the method is phage library-based assay, comprising administering a phage library, or portion thereof, to the first chamber. In one embodiment, the method comprises detecting which surface peptides are present in the collected contents of the second chamber. In one embodiment, the mucus-penetrating peptides in the collected contents are identified by isolating the phage and identifying the peptide(s) expressed on the isolated phage by one or more of plaque counting, phage amplification, and sequencing the phage DNA to identify the phage-presented peptide which mediates mucus penetration. In one embodiment, the mucus-penetrating peptides in the collected contents are identified by collecting the DNA in the second chamber and analyzing the collected DNA to identify the mucus-penetrating peptides. Analysis of the collected DNA, collected either directly from the second chamber or from the isolated phage, can be conducted by one or more of DNA sequencing, next generation sequencing, Sanger sequencing, high throughput sequencing, nanopore sequencing, droplet coupled next generation sequencing, digital PCR with next generation sequencing, DNA microarrays, optical mapping, and NanoString. Further, the collected DNA can be analyzed using any appropriate methodology developed in the future (Goodwin et al., 2016, Nature Reviews Genetics, 17: 333-351).

### Compositions

In one aspect, the present invention provides a composition comprising one or more mucus-penetrating peptides. In certain embodiments, the mucus-penetrating peptides are identified by way of the screening method described elsewhere herein.

In certain embodiments, the composition comprises the combination of (1) one or more mucus-penetrating peptides, and (2) an agent desired to be transported through a mucosal barrier. Exemplary agents include, but is not limited to, a therapeutic agent, prophylactic agent, diagnostic agent, imaging agent, contrast agent, microparticle, nanoparticle, and the like. In certain embodiments, the mucus-penetrating peptide facilitates transport of the agent through the barrier.

In certain embodiments, the composition comprises a fusion construct comprising one or more mucus-penetrating peptides fused, linked, or conjugated to an agent. In certain embodiments, the one or more mucus-penetrating peptides are able to transport the fusion construct through a mucosal barrier in order to access a target site located on the other side of a mucosal barrier.

In one embodiment, the one or more mucus-penetrating peptides comprises one or more peptides selected from SEQ ID NOs: 1-28, as depicted in Table 1. In certain instances, the one or more mucus-penetrating peptides are hydrophilic. In certain instances, the one or more mucus-penetrating peptides are enriched in hydrophilic amino acid residues.

TABLE 1

| | |
|---|---|
| SEQ ID NO: 1 | LTAQPST |
| SEQ ID NO: 2 | ACTVRTSADC |
| SEQ ID NO: 3 | VNRSSLY |
| SEQ ID NO: 4 | GETRAPL |
| SEQ ID NO: 5 | APTAVSK |
| SEQ ID NO: 6 | TPHPLRL |
| SEQ ID NO: 7 | APKQSLE |
| SEQ ID NO: 8 | VSTPSTP |
| SEQ ID NO: 9 | GGLSSRP |
| SEQ ID NO: 10 | YPSPWGY |
| SEQ ID NO: 11 | TLNRVPN |
| SEQ ID NO: 12 | GVPTALP |
| SEQ ID NO: 13 | QLVYPAP |
| SEQ ID NO: 14 | SSQLSRP |
| SEQ ID NO: 15 | LGTSMQL |
| SEQ ID NO: 16 | SLGPSPG |
| SEQ ID NO: 17 | ISLPSPT |
| SEQ ID NO: 18 | MISSNSS |
| SEQ ID NO: 19 | YNSPTHH |
| SEQ ID NO: 20 | SGTHHKA |
| SEQ ID NO: 21 | TNTMTRA |
| SEQ ID NO: 22 | KPFPPMK |
| SEQ ID NO: 23 | ETTHLTG |
| SEQ ID NO: 24 | SPHDVAYD |
| SEQ ID NO: 25 | QLKPLEF |
| SEQ ID NO: 26 | LPLWEVY |
| SEQ ID NO: 27 | TVRTSAD |
| SEQ ID NO: 28 | NTGSPYE |

The peptides of the composition may comprise amino acid residues that are of the L- or D-enantiomer. The peptides of the present invention further include conservative variants of the peptides herein described, according to another embodiment. As used herein, a “conservative variant” refers to alterations in the amino acid sequence that do not substantially and adversely affect the binding or association capacity of the peptide. A substitution, insertion or deletion is said to adversely affect the peptide when the altered sequence prevents, reduces, or disrupts a function or activity associated with the peptide. For example, the overall charge, structure or hydrophobic-hydrophilic properties of the peptide can be altered without adversely affecting an activity. Accordingly, the amino acid sequence can be altered, for example to render the peptide more hydrophobic or hydrophilic, without adversely affecting the activities of the peptide.

These variants, though possessing a slightly different amino acid sequence than those recited elsewhere herein, will still have the same or similar properties associated with any of the peptides discussed herein. Ordinarily, the conservative substitution variants, will have an amino acid sequence having at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity with any of the peptides discussed elsewhere herein.

In certain embodiments, the composition comprises a fragment of one or more of the peptides discussed elsewhere herein. For example, in certain embodiments, the fragment comprises 2 or more, 3 or more, 4 or more, 5 or more, 6 or more, 7 or more, 8 or more, 9 or more, 10 or more, 15 or more, or 20 or more amino acid residues of one of any of the peptides discussed elsewhere herein.

The peptide may comprise one or more hydrophilic residues. For example, in certain embodiments, the peptide comprises 1 or more, 2 or more, 3 or more, 4 or more, 5 or more, 6 or more, 7 or more, 8 or more, 9 or more, 10 or more, 15 or more, or 20 or more hydrophilic amino acid residues. The hydrophilic amino acid residues may be consecutive or non-consecutive. In certain embodiments, the peptide is enriched in hydrophilic residues. For example, in certain embodiments, the peptide comprises 40% or more, 50% or more, 60% or more, 70% or more, 75% or more, 80% or more, 85% or more, 90% or more, or 95% or more hydrophilic residues.

In some embodiments, the composition, for example the mucus-penetrating peptide of the composition, are able to associate with (or bind to) specific sequences of DNA or other proteins. These peptides may be able to bind, for example, to DNA or other proteins with high affinity and selectivity. As used herein, the term “bind” or “binding” refers to the specific association or other specific interaction between two molecular species, such as, but not limited to, protein-DNA interactions and protein-protein interactions, for example, the specific association between proteins and their DNA targets, receptors and their ligands, enzymes and their substrates, etc. Such binding may be specific or non-specific, and can involve various noncovalent interactions, such as including hydrogen bonding, metal coordination, hydrophobic forces, van der Waals forces, pi-pi interactions, and/or electrostatic effects. It is contemplated that such association may be mediated through specific sites on each of two (or more) interacting molecular species. Binding can be mediated by structural and/or energetic components. In some cases, the latter will comprise the interaction of molecules with opposite charges.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Peptide Analogs

The present invention relates to peptide analogs of peptides comprising one or more of SEQ ID NOs: 1-28, or any another peptide appropriate for use with the invention and uses thereof. For example, in certain instances the invention provides peptides and peptide analogs based on fragments, analogs, or derivatives of peptides comprising one or more of SEQ ID NOs: 1-28, where the peptides and peptide analogs exhibit desirable properties. In one embodiment, the invention provides compositions comprising peptides and analogs, fragments, and derivatives thereof that exhibit one or more of improved solubility, half-life, bioavailability, reduced renal clearance and the like compared to SEQ ID NOs: 1-28. In one embodiment, the invention provides compositions comprising peptides and analogs, fragments, and derivatives thereof that exhibit one or more of improved solubility, half-life, bioavailability, reduced renal clearance and the like compared to SEQ ID NOs: 1-28.

A peptide or chimeric protein of the invention may be phosphorylated using conventional methods such as the method described in Reedijk et al. (The EMBO Journal 11(4):1365, 1992).

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

In one embodiment, the subject compositions are peptidomimetics of the peptides of the invention, for example, peptidomimetics of peptides comprising one or more of SEQ ID NOs: 1-28. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and nonpeptide synthetic structures; peptidomimetics may be useful, therefore, in delineating pharmacophores and in helping to translate peptides into nonpeptide compounds with the activity of the parent peptides.

The peptidomimetics of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALYS), could be modified with an amine specific photoaffinity label.

Fusion Constructs

A peptide of the invention may be fused with, linked to, or conjugated with other molecules, to prepare fusion constructs. This may be accomplished, for example, by the synthesis of N-terminal or C-terminal fusion constructs provided that the resulting fusion construct retains the mucus-penetrating function of the peptide.

In one embodiment, the composition comprises a construct comprising one or more agents fused with, linked to, or conjugated with, one or more mucus-penetrating peptides described elsewhere herein. The one or more agents may include, but is not limited to, therapeutic agents, prophylactic agents, chemotherapeutic agents, diagnostic agents, imaging agents, radiosensitizing agents, contrast agents, drug delivery vehicles, liposomes, polymerosomes, micelles, microparticles, nanoparticles, and the like. Exemplary agents include, but is not limited to, peptides, nucleic acid molecule, antisense nucleic acid molecules, small molecule drugs, organic compounds, inorganic compounds, antibodies, vitamins, hormones, cytokines, growth factors, detectable labels, quantum dots, and the like.

The mucus-penetrating peptide may be linked to the agent using any methodology known in the art, including, but not limited to, covalent linkage, noncovalent linkage, crosslinking, peptide linkers, nucleotide linkers, and the like. Linkages can include but not limited to isothiocyanate, NHS, haloacetyl, maleimide or other thiolation linkers, disulfide, glucuronide linkage, acid sensitive linkers (e.g. hydrazone), enzyme cleavable linkers (Val-Cit dipeptide, linkages cleavable by matrix metalloproteinases and cathespin proteases), and click chemistry linkages.

Exemplary therapeutic agents include, but are not limited to analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, bronchodialators, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like.

In one embodiment, the therapeutic agent comprises a mucus degrading agent. A mucus degrading agent refers to a substance which increases the rate of mucus clearance when administered to a subject. Mucus degrading agents are known in the art. See, for example, Hanes, J. et al. Gene Delivery to the Lung. in Pharmaceutical Inhalation Aerosol Technology, Marcel Dekker, Inc., New York: 489-539 (2003). Examples of mucus degrading agents include N-acetylcysteine (NAC), which cleaves disulfide and sulfhydryl bonds present in mucin. Other mucus degrading agents include mugwort, bromelain, papain, clerodendrum, acetylcysteine, bromhexine, carbocisteine, eprazinone, mesna, ambroxol, sobrerol, domiodol, denufosol, letosteine, stepronin, tiopronin, gelsolin, thymosin (34, neltenexine, erdosteine, and various DNases including rhDNase.

Transported Agents

In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule therapeutic agent of the composition comprises an analog or derivative of a therapeutic agent described herein. In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present invention can be used to treat a disease or disorder.

In one embodiment, the small molecule therapeutic agents described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, or miRNA molecule. In one embodiment, the therapeutic agent is an isolated nucleic acid encoding a therapeutic peptide. For example, in certain embodiments, the present invention provides a gene therapy composition comprising one or more mucus-penetrating peptides described herein.

In some instances the therapeutic agent is an siRNA, miRNA, or antisense molecule, which inhibits a targeted nucleic acid. In one embodiment, the nucleic acid comprises a promoter/regulatory sequence such that the nucleic acid is preferably capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In one embodiment, the therapeutic agent is an siRNA RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of PTPN22 using RNAi technology.

In one embodiment, the therapeutic agent is a short hairpin RNA (shRNA) therapeutic agent. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

In one embodiment of the invention, an antisense nucleic is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, Calif.) or they may be genetically expressed from DNA encoding them.

In one embodiment, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In one embodiment, the therapeutic agent comprises a gRNA or a nucleic acid molecule encoding a gRNA. In one embodiment, the therapeutic agents comprises a Cas peptide or a nucleic acid molecule encoding a Cas peptide.

In other related aspects, the therapeutic agent includes an isolated peptide that modulates a target. For example, in one embodiment, the peptide of the invention inhibits or activates a target directly by binding to the target thereby modulating the normal functional activity of the target. In another embodiment, the peptide of the invention modulates the target by competing with endogenous proteins. In yet another embodiment, the peptide of the invention modulates the activity of the target by acting as a transdominant negative mutant.

In one embodiment, the therapeutic agent is an antibody. In certain embodiments, the antibody can inhibit a target to provide a beneficial effect. The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain FV molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

Imaging agents are materials that allow for visualization after exposure to a cell or tissue. Visualization includes imaging for the naked eye, as well as imaging that requires detecting with instruments or detecting information not normally visible to the eye, and includes imaging that requires detecting of photons, sound or other energy quanta. Examples include stains, vital dyes, fluorescent markers, radioactive markers, enzymes or plasmid constructs encoding markers or enzymes. Many materials and methods for imaging and targeting that may be used in the composition of the invention are provided in the Handbook of Targeted delivery of Imaging Agents, Torchilin, ed. (1995) CRC Press, Boca Raton, Fla. Visualization based on molecular imaging typically involves detecting biological processes or biological molecules at a tissue, cell, or molecular level. Molecular imaging can be used to assess specific targets for gene therapies, cell-based therapies, and to visualize pathological conditions as a diagnostic or research tool. Imaging agents that are able to be delivered intracellularly are particularly useful because such agents can be used to assess intracellular activities or conditions. Suitable imaging agents include, for example, fluorescent molecules, labeled antibodies, labeled avidin:biotin binding agents, colloidal metals (e.g., gold, silver), reporter enzymes (e.g., horseradish peroxidase), superparamagnetic transferrin, second reporter systems (e.g., tyrosinase), and paramagnetic chelates. In some embodiments, the imaging agent is a Magnetic resonance imaging contrast agent. Examples of Magnetic resonance imaging contrast agents include, but are not limited to, 1,4,7,10-tetraazacyclododecane-N,N,N″N′″-tetracetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (DOTA) and derivatives thereof (see U.S. Pat. Nos. 5,188,816, 5,219,553, and 5,358,704). In some embodiments, the imaging agent is an X-Ray contrast agent. X-ray contrast agents already known in the art include a number of halogenated derivatives, especially iodinated derivatives, of 5-amino-isophthalic acid.

Exemplary detectable labels include, but are not limited to biotin, an enzyme, an epitope, a radionuclide, a fluorescent molecule, and the like.

In certain embodiments, the composition comprises an imaging agent that may be further attached to a detectable label (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. A composition including such a moiety may be used as an imaging agent and be administered in an amount effective for diagnostic use in a mammal such as a human. In this manner, the localization and accumulation of the imaging agent can be detected. The localization and accumulation of the imaging agent may be detected by radioscintiography, nuclear magnetic resonance imaging, computed tomography, or positron emission tomography. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, preferably 1-10 millicuries, most often 2-5 millicuries are administered. Thus, compositions according to the present invention useful as imaging agents comprising a targeting moiety conjugated to a radioactive moiety comprise 0.1-100 millicuries, in some embodiments preferably 1-10 millicuries, in some embodiments preferably 2-5 millicuries, in some embodiments more preferably 1-5 millicuries.

The means of detection used to detect the label is dependent of the nature of the label used and the nature of the biological sample used, and may also include fluorescence polarization, high performance liquid chromatography, antibody capture, gel electrophoresis, differential precipitation, organic extraction, size exclusion chromatography, fluorescence microscopy, or fluorescence activated cell sorting (FACS) assay.

In certain embodiments, the peptide is fused to, linked to, a drug delivery vehicle, wherein the vehicle comprises an agent, for example, a therapeutic agent, prophylactic agent, imaging agent, or contrast agent.

In certain embodiments, the one or more agents may be linked to the one or more mucus-penetrating peptides using any known methodology known in the art. The one or more agents may be directly or indirectly linked or conjugated to the one or more mucus-penetrating peptides. For example, in certain embodiments, the one or more agents may be linked to the one or more mucus-penetrating peptides via a linker peptide sequence.

In one embodiment, the composition comprises one or more mucus-penetrating peptide and one or more targeting moieties. For example, the one or more targeting moieties can be any moiety recognized by a transmembrane or intracellular receptor protein. In one embodiment, a targeting moiety is a ligand. The ligand, according to the present invention, preferentially binds to and/or internalizes into a cell in which the attached nucleic acid by way of the interaction with the densely packed cationic amino acid residues enters the cell. A ligand is usually a member of a binding pair where the second member is present on, or in a target cell, or in a tissue comprising the target cell. Examples of ligands suitable for the present invention are: folic acid, protein (e.g., transferrin), growth factor, enzyme, peptide, receptor, antibody or antibody fragment, such as Fab′, Fv, single chain Fv, single-domain antibody, or any other polypeptide comprising antigen-binding sequences (CDRs) of an antibody molecule. In one embodiment, the targeting moiety specifically interacts with a growth factor receptor, an angiogenic factor receptor, a transferrin receptor, a cell adhesion molecule, or a vitamin receptor. The choice of targeting moiety depends upon the type and number of ligands that define the surface of a target cell. For example, the targeting moiety may be chosen to recognize a ligand that acts as a cell surface marker on target cells associated with a particular disease state. Thus, examples of cell surface markers that may act as ligands for the targeting moiety in the composition of the invention include those associated with viral, bacterial and parasitic infections, autoimmune disease and cancer cells.

The one or more agents may comprise an antibiotic, such as tobramycin, colistin, or aztreonam. The one or more agents may comprise one or more inhaled corticosteroids, such as flunisolide, triamcinolone acetonide, beclomethasone dipropionate, mometasone, budesonide, ciclesonide, or fluticasone propionate. The one or more agents may comprise an anti-inflammatory antibiotic, such as erythromycin, azithromycin, or clarithromycin. The one or more agents may comprise chemotherapeutic agents, and anti-proliferative agents.

Nucleic Acid

The present invention further provides, in another embodiment, nucleic acid molecules that encode any of the amino acid sequences discussed herein. As used herein, “nucleic acid” includes cDNA and mRNA, as well as nucleic acids based on alternative backbones or including alternative bases whether derived from natural sources or synthesized. Those of ordinary skill in the art, given an amino acid sequence, will be able to generate corresponding nucleic acid sequences that can be used to generate the amino acid sequence, using no more than routine skill.

In one embodiment, the composition comprises a nucleic acid molecule encoding a peptide comprising one or more of SEQ ID NOs: 1-28. In one embodiment, the nucleic acid molecule encodes a peptide comprising an amino acid sequence having substantial homology to any one of SEQ ID NOs: 1-28. For example, in one embodiment, the nucleic acid molecule encodes a peptide comprising an amino acid sequence having at least 75%, at least 80%, at least 85%, at least 90%, at least 95%, at least 98%, or at least 99% sequence identity with any of the peptides discussed elsewhere herein.

For example, in one embodiment the nucleic acid molecule encodes a peptide comprising an amino acid molecule comprising 2 or more, 3 or more, 4 or more, 5 or more, 6 or more, 7 or more, 8 or more, 9 or more, 10 or more, 15 or more, or 20 or more amino acid residues of one of any of the peptides discussed elsewhere herein.

Modifications to the primary structure itself by deletion, addition, or alteration of the amino acids incorporated into the peptide sequence during translation can be made without destroying the activity of the peptide. Such substitutions or other alterations result in peptides having an amino acid sequence encoded by a nucleic acid falling within the contemplated scope of the present invention.

The present invention further provides, in some embodiments, recombinant DNA molecules that contain a coding sequence. As used herein, a “recombinant DNA molecule” is a DNA molecule that has been subjected to molecular manipulation. Methods for generating recombinant DNA molecules are well known in the art, for example, see Sambrook et al., 2012, Molecular Cloning: A Laboratory Manual, Cold Spring Harbor Laboratory, New York. In some recombinant DNA molecules, a coding DNA sequence is operably linked to expression control sequences and vector sequences.

The choice of vector and expression control sequences to which one of the peptide family encoding sequences of the present invention is operably linked depends directly, as is well known in the art, on the functional properties desired (e.g., protein expression, and the host cell to be transformed). A vector of the present invention may be at least capable of directing the replication or insertion into the host chromosome, and preferably also expression, of the structural gene included in the recombinant DNA molecule.

Expression control elements that are used for regulating the expression of an operably linked protein encoding sequence are known in the art and include, but are not limited to, inducible promoters, constitutive promoters, secretion signals, and other regulatory elements. In some embodiments, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extra-chromosomal in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical of bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Any suitable prokaryotic host can be used to express a recombinant DNA molecule encoding a peptide of the invention.

Expression vectors compatible with eukaryotic cells, including those compatible with vertebrate cells, can also be used to form recombinant DNA molecules that contain a coding sequence. Eukaryotic cell expression vectors are well known in the art and are available from several commercial sources. Typically, such vectors are provided containing convenient restriction sites for insertion of the desired DNA segment.

Eukaryotic cell expression vectors used to construct the recombinant DNA molecules of the present invention may further include a selectable marker that is effective in a eukaryotic cell, such as a drug resistance selection marker. An example drug resistance marker is the gene whose expression results in neomycin resistance, i.e., the neomycin phosphotransferase (neo) gene. Alternatively, the selectable marker can be present on a separate plasmid, the two vectors introduced by co-transfection of the host cell, and transfectants selected by culturing in the appropriate drug for the selectable marker.

The present invention further provides, in yet another embodiment, host cells transformed with a nucleic acid molecule that encodes a peptide of the present invention. The host cell can be either prokaryotic or eukaryotic. Eukaryotic cells useful for expression of a peptide of the invention are not limited, so long as the cell line is compatible with cell culture methods and compatible with the propagation of the expression vector and expression of the gene product.

Transformation of appropriate cell hosts with a recombinant DNA molecule encoding a peptide of the present invention is accomplished by well-known methods that typically depend on the type of vector used and host system employed. With regard to transformation of prokaryotic host cells, electroporation and salt treatment methods can be employed (see, for example, Sambrook et al., 2012, Molecular Cloning: A Laboratory Manual, Cold Spring Harbor Laboratory Press). With regard to transformation of vertebrate cells with vectors containing recombinant DNA, electroporation, cationic lipid or salt treatment methods can be employed (see, for example, Graham et al., (1973) Virology 52, 456-467; Wigler et al., (1979) Proc. Natl. Acad. Sci. USA 76, 1373-1376).

Successfully transformed cells can be identified by well-known techniques including the selection for a selectable marker. For example, cells resulting from the introduction of a recombinant DNA of the present invention can be cloned to produce single colonies. Cells from those colonies can be harvested, lysed and their DNA content examined for the presence of the recombinant DNA using a method such as that described by Southern (1975) J. MoI. Biol. 98, 503-517, or the peptides produced from the cell assayed via an immunological method.

The present invention further provides, in still another embodiment, methods for producing a peptide of the invention using nucleic acid molecules herein described. In general terms, the production of a recombinant form of a peptide typically involves the following steps: a nucleic acid molecule is obtained that encodes a peptide of the invention.

The nucleic acid molecule may then be placed in operable linkage with suitable control sequences, as described above, to form an expression unit containing the peptide open reading frame. The expression unit is used to transform a suitable host and the transformed host is cultured under conditions that allow the production of the recombinant peptide. Optionally the recombinant peptide is isolated from the medium or from the cells; recovery and purification of the peptide may not be necessary in some instances where some impurities may be tolerated.

Each of the foregoing steps can be done in a variety of ways. The construction of expression vectors that are operable in a variety of hosts is accomplished using appropriate replicons and control sequences, as set forth above. The control sequences, expression vectors, and transformation methods are dependent on the type of host cell used to express the gene. Suitable restriction sites, if not normally available, can be added to the ends of the coding sequence, so as to provide an excisable gene to insert into these vectors. An artisan of ordinary skill in the art can readily adapt any host/expression system known in the art for use with the nucleic acid molecules of the invention to produce a recombinant peptide.

Genetically-Modified Cell

In one embodiment, the composition comprises a cell modified to express one or more mucus-penetrating peptide described elsewhere herein. For example, in one embodiment, the cell is modified to express one or more peptides comprising one or more of SEQ ID NOs: 1-28, variants thereof, or fragments thereof. In certain embodiments, the cell is genetically modified by introducing to the cell one or more nucleic acid molecules encoding the one or more mucus-penetrating peptides described elsewhere herein. In certain embodiments, the cell is modified to express the one or more mucus-penetrating peptides on its surface, thereby allowing for the transport of the cell through a mucosal barrier. In certain embodiments, the modified cell is a therapeutic cell, including but not limited to an immune cell, stem cell, or the like. In one embodiment, the cell is a prokaryotic cell, for example a bacterial cell.

### Methods

In certain embodiments, the present invention relates to methods of delivering an agent through a mucosal barrier. In one embodiment, the method comprises administering to a subject a composition comprising one or more mucus-penetrating peptides described elsewhere herein. The method may be used to deliver the one or more agents through mucosal barriers present at gastrointestinal, vaginal, rectal, respiratory, nasal, and ophthalmic tissues.

In one embodiment, the method comprises co-administering (1) one or more mucus-penetrating peptides described herein and (2) an agent to be transported. As described herein, the peptides described herein facilitates the transport of the agent through a barrier.

In one embodiment, the composition comprises a fusion construct comprising one or more mucus-penetrating peptides linked to, fused to, or conjugated to, one or more agents. In one embodiment, the one or more mucus-penetrating peptides delivers the one or more agents through a mucosal battier to a target site located on the other side of a mucosal barrier.

In certain embodiments, the method uses the compositions described herein for enhanced oral delivery of an agent, such a therapeutic or prophylactic agent, where the mucus-penetrating peptide enhances delivery of the agent through the gastrointestinal mucosal barrier to the bloodstream.

In one embodiment, the present invention provides a method of treating or preventing a disease or disorder comprising administering to a subject a composition comprising a therapeutic or prophylactic agent fused to, linked to, or conjugated to one or more mucus-penetrating peptides. In one embodiment, the therapeutic or prophylactic agent is suitable to treat or prevent the disease or disorder. The presently described method is useful against any disease or disorder in which transport of the agent through a mucosal barrier would be beneficial. For example, the present method may be used to treat or prevent any disease or disorder where a therapeutic agent must pass through a mucosal barrier to reach a target site. Exemplary diseases and disorders include, but are not limited to, ocular-based diseases, cystic fibrosis, chronic obstructive pulmonary disease and their associated infections, diseases of the GI tract, blood-borne diseases, bacterial infections, viral infections, cancer and autoimmune disorders. It will be appreciated that the peptides of the invention may be administered to a subject either alone, or in conjunction with another therapeutic agent. In one embodiment, the peptides of the invention are administered to a subject in combination with an anti-cancer therapy.

In a particular embodiment, the invention provides a method of treating cystic fibrosis, which is associated with abnormal mucus production. The method comprises administering to the subject a composition comprising one or more mucus-penetrating peptides and a therapeutic agent suitable to treat cystic fibrosis or cystic fibrosis-associated conditions (e.g., infection). However, the present invention is not limited to cystic fibrosis, but rather encompasses the use of the compositions described herein in any pulmonary disease or disorder, including but not limited to, bronchitis, asthma, chronic obstructive pulmonary disease (COPD), and emphysema, where delivery of an active agent through the mucus to the lung is beneficial.

In certain embodiments, the invention provides a method of treating or preventing a disease or disorder of the female reproductive system, including but not limited to, vaginal cancer, cervical cancer, pelvic inflammatory disease, endometriosis, uterine fibroids, polycystic ovary syndrome, ovarian cysts, vulvovaginits, infertility, and sexually transmitted diseases, where the compositions described herein can be used to transport an active agent through the mucosal barrier in the vagina.

In one embodiment, the present invention provides a method for ophthalmic delivery of an agent comprising administering to the surface of the eye a composition comprising a therapeutic or prophylactic agent fused to, linked to, or conjugated to one or more mucus-penetrating peptides. For example, in certain aspects, the mucus-penetrating peptides described herein allows for diffusion of the agent through the eye to a desired treatment site.

In one embodiment, the present invention provides an imaging method comprising administering to a subject a composition comprising an imaging agent fused to, linked to, or conjugated to one or more mucus-penetrating peptides.

In one embodiment, the present invention provides a diagnostic method comprising administering to a subject a composition comprising an diagnostic or imaging agent fused to, linked to, or conjugated to one or more mucus-penetrating peptides. For example, the compositions described herein allow for the delivery of the diagnostic or imaging agent through a mucosal barrier, thereby allowing the diagnostic or imaging agent to access a site of interest.

### Pharmaceutical Compositions and Methods of Treatment

The methods of the invention thus encompass the use of pharmaceutical compositions comprising one or more mucus-penetrating peptides of the invention to practice the methods of the invention. The pharmaceutical compositions useful for practicing the invention may be administered to deliver a dose of from 100 ng/kg/day to 100 mg/kg/day. In one embodiment, the invention envisions administration of a dose which results in a concentration of the compound of the present invention from 1 μM to 10 μM in a mammal.

Typically, dosages which may be administered in a method of the invention to a mammal, preferably a human, range in amount from 0.5 μg to about 50 mg per kilogram of body weight of the mammal. The precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

The peptides and constructs of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein.

Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. Aerosols for the delivery of pharmaceutical compositions to the respiratory tract are known in the art. The term aerosol as used herein refers to any preparation of a fine mist of solid or liquid particles suspended in a gas. In some cases, the gas may be a propellant; however, this is not required. Aerosols may be produced using a number of standard techniques, including as ultrasonication or high pressure treatment. In certain instances, a dry powder or liquid formulation is formulated into aerosol formulations using one or more propellants. Suitable propellants include air, hydrocarbons, such as pentane, isopentane, butane, isobutane, propane and ethane, carbon dioxide, chlorofluorocarbons, fluorocarbons, hydrofluroalkanes (HFA), and combinations thereof.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for treatment of a disease. Using a wax/pH-sensitive polymer mix, a gastric insoluble composition may be obtained in which the active ingredient is entrapped, ensuring its delayed release.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

A pharmaceutical composition of the invention may be prepared, packaged, or sold in a formulation suitable for vaginal administration. With respect to the vaginal or perivaginal administration of the compounds of the invention, dosage forms may include vaginal suppositories, creams, ointments, liquid formulations, pessaries, tampons, gels, pastes, foams or sprays. The suppository, solution, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention may be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit may be fabricated to disintegrate rapidly or over a period of several hours. The time period for complete disintegration may be in the range of from about 10 minutes to about 6 hours, e.g., less than about 3 hours.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject.

Douche preparations may further comprise various additional ingredients including, but not limited to, antioxidants, antibiotics, antifungal agents, and preservatives.

In some cases, the one or more active agents are delivered into the lungs by inhalation of an aerosolized pharmaceutical formulation. Inhalation can occur through the nose and/or the mouth of the patient. Administration can occur by self-administration of the formulation while inhaling, or by administration of the formulation via a respirator to a patient on a respirator. In some cases, a device is used to administer the formulations to the lungs. Suitable devices include, but are not limited to, dry powder inhalers, pressurized metered dose inhalers, nebulizers, and electrohydrodynamic aerosol devices.

Dry powder formulations can be administered to the lungs using a dry powder inhaler (DPI). DPI devices typically use a mechanism such as a burst of gas to create a cloud of dry powder inside a container, which can then be inhaled by the subject. In a dry powder inhaler, the dose to be administered is stored in the form of a non-pressurized dry powder and, on actuation of the inhaler, the particles of the powder are inhaled by the subject. In some cases, a compressed gas (i.e., propellant) may be used to dispense the powder, similar to pressurized metered dose inhalers (pMDIs). In some cases, the DPI may be breath actuated, meaning that an aerosol is created in precise response to inspiration. Typically, dry powder inhalers administer a dose of less than a few tens of milligrams per inhalation to avoid provocation of cough.

DPIs function via a variety of mechanical means to administer formulations to the lungs. In some DPIs, a doctor blade or shutter slides across the dry powder formulation contained in a reservoir, culling the formulation into a flowpath whereby the subject can inhale the powder in a single breath. In other DPIs, the dry powder formulation is packaged in a preformed dosage form, such as a blister, tabule, tablet, or gelcap, which is pierced, crushed, or otherwise unsealed to release the dry powder formulation into a flowpath for subsequent inhalation. Still others DPIs release the dry powder formulation into a chamber or capsule and use mechanical or electrical agitators to keep the dry powder formulation suspended in the air until the patient inhales.

Dry powder formulations may be packaged in various forms, such as a loose powder, cake, or pressed shape for insertion in to the reservoir of a DPI.

Liquid formulations can be administered to the lungs of a subject using a pressurized metered dose inhaler (pMDI). Pressurized Metered Dose Inhalers (pMDIs) generally include at least two components: a canister in which the liquid formulation is held under pressure in combination with one or more propellants, and a receptacle used to hold and actuate the canister. The canister may contain a single or multiple doses of the formulation. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the liquid formulation is atomized, forming an aerosol. pMDIs typically employ one or more propellants to pressurize the contents of the canister and to propel the liquid formulation out of the receptacle outlet, forming an aerosol. Any suitable propellants, including those discussed above, may be utilized. The propellant may take a variety of forms. For example, the propellant may be a compressed gas or a liquefied gas. Chlorofluorocarbons (CFC) were once commonly used as liquid propellants, but have now been banned. They have been replaced by the now widely accepted hydrofluororalkane (HFA) propellants. In some cases, the subject administers an aerosolized formulation by manually discharging the aerosolized formulation from the pMDI in coordination with inspiration. In this way, the aerosolized formulation is entrained within the inspiratory air flow and conveyed to the lungs. In other cases, a breath-actuated trigger may be employed that simultaneously discharges a dose of the formulation upon sensing inhalation. These devices, which discharge the aerosol formulation when the user begins to inhale, are known as breath-actuated pressurized metered dose inhalers (baMDls).

Liquid formulations can also be administered using a nebulizer. Nebulizers are liquid aerosol generators that convert the liquid formulation described able, usually aqueous-based compositions, into mists or clouds of small droplets, preferably having diameters less than 5 microns mass median aerodynamic diameter, which can be inhaled into the lower respiratory tract. This process is called atomization. The droplets carry the one Or more active agents into the nose, upper airways or deep lungs when the aerosol cloud is inhaled. Any type of nebulizer may be used to administer the formulation to a patient, including, but not limited to pneumatic (jet) nebulizers and electromechanical nebulizers.

Pneumatic (jet) nebulizers use a pressurized gas supply as a driving force for atomization of the liquid formulation. Compressed gas is delivered through a nozzle or jet to create a low pressure field which entrains a surrounding liquid formulation and shears it into a thin film or filaments. The film or filaments are unstable and break up into small droplets that are carried by the compressed gas flow into the inspiratory breath. Baffles inserted into the droplet plume screen out the larger droplets and return them to the bulk liquid reservoir.

Electromechanical nebulizers use electrically generated mechanical force to atomize liquid formulations. The electromechanical driving force can be applied, for example, by vibrating the liquid formulation at ultrasonic frequencies, or by forcing the bulk liquid through small holes in a thin film. The forces generate thin liquid films or filament streams which break up into small droplets to form a slow moving aerosol stream which can be entrained in an inspiratory flow.

In some cases, the electromechanical nebulizer is an ultrasonic nebulizer, in which the liquid formulation is coupled to a vibrator oscillating at frequencies in the ultrasonic range. The coupling is achieved by placing the liquid in direct contact with the vibrator such as a plate or ring in a holding cup, or by placing large droplets on a solid vibrating projector (a horn). The vibrations generate circular standing films which break up into droplets at their edges to atomize the liquid formulation.

In some cases, the electromechanical nebulizer is a mesh nebulizer, in which the liquid formulation is driven through a mesh or membrane with small holes ranging from 2 to 8 microns in diameter, to generate thin filaments which break up into small droplets. In certain designs, the liquid formulation is forced through the mesh by applying pressure with a solenoid piston driver, or by sandwiching the liquid between a piezoelectrically vibrated plate and the mesh, which results in a oscillatory pumping action. In other cases, the mesh vibrates back and forth through a standing column of the liquid to pump it through the holes

Liquid formulations can also be administered using an electrohydrodynamic (EHD) aerosol device. EHD aerosol devices use electrical energy to aerosolize liquid drug solutions or suspensions. Examples of EHD aerosol devices are known in the art. See, for example, U.S. Pat. No. 4,765,539 to Noakes et al. and U.S. Pat. No. 4,962,885 to Coffee, R. A.

In certain embodiments, the compositions are formulated for ocular or periocular delivery. Ophthalmic compositions can be in the form of solutions. Solutions can be administered topically by applying them to the cul-de-sac of the eye from a dropper controlled bottle or dispenser. A typical dose regimen for an adult human may range from about 2 to about 8 drops per day, applied at bed-time or throughout the day. Dosages for adult humans may, however, be higher, in which case the drops are administered by “bunching”, e.g., 5 doses administered over a 5 minute period, repeated about 4 times daily. A topical solution in accordance with one embodiment of the invention comprises a therapeutic dose of a composition described herien in an artificial tear formulation. Typically, artificial tear compositions contain ionic components found in normal human tear film, as well as various combinations of one or more of tonicity agents (e.g., soluble salts, such as Na, Ca, K, and Mg chlorides, and dextrose and sorbitol), buffers (e.g., alkali metal phosphate buffers), viscosity/lubricating agents (e.g., alkyl and hydroxyalkyl celluloses, dextrans, polyacrylamides), nonionic surfactants, sequestering agents (e.g., disodium edetate, citric acid, and sodium citrate), and preservatives (e.g., benzalkonium chloride, and thimerosal). In one embodiment, artificial tear compositions are preservative free. The quantities and relative proportions of each of these components incorporated into an artificial tear composition are readily determinable by the skilled formulation chemist. The ionic species bicarbonate is used in artificial tear compositions, e.g., U.S. Pat. No. 5,403,598 and Ubels, J L, et al, Arch. Ophthalmol. 1995, 113: 371-8.

Alternatively, the compositions described herein can be in the form of ophthalmic ointments. Ophthalmic ointments have the benefit of providing prolonged drug contact time with the eye surface. Ophthalmic ointments will generally include a base comprised of, for example, white petrolatum and mineral oil, often with anhydrous lanolin, polyethylene-mineral oil gel, and other substances recognized by the formulation chemist as being non-irritating to the eye, which permit diffusion of the drug into the ocular fluid, and which retain activity of the medicament for a reasonable period of time under storage conditions.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Development of a Biomolecule-Screening Assay to Identify Mucus-Penetrating Peptides

Described herein is a peptide-based strategy to identify mucus-penetrating formulations and understand physicochemical properties for improved mucosal transport. Previously, phage display has been utilized as a technology to discover peptides with selective affinity for a broad spectrum of targets including antibodies, epitopes, small molecules and synthetic materials (Ghosh et al., 2014, Proc Natl Acad Sci USA 111, 13948-13953; Ghosh et al., 2005, Journal of Virology 79, 13667). The strategy described herein leverages the diversity of these large, engineered libraries of random peptides (10⁻⁸-10⁻⁹) such that this collection of “peptide-based formulations” can be screened to identify peptides with “stealth-like” properties for enhanced mucosal transport. Phage libraries with 2×10⁹ diversity were incubated on 20% w/v mucin in a donating reservoir of a transwell chamber with a polyethylene terephthalate semipermeable membrane. Phage that penetrated through the mucin were collected in the bottom receiving reservoir at 0.25 and 6 hours. Then, collected phage were grown in XL-1 E. coli to amplify copies of penetrating phage particles. This screening process was iterated for several rounds to collapse the library to few phage-presenting peptides most able to rapidly penetrate the mucin barrier. After, 2 or 3 rounds of sequencing, phage were plated, overlaid on agar and incubated overnight. Twenty plaques (i.e. individual phage) were grown in liquid culture and phage DNA was isolated for sequencing.

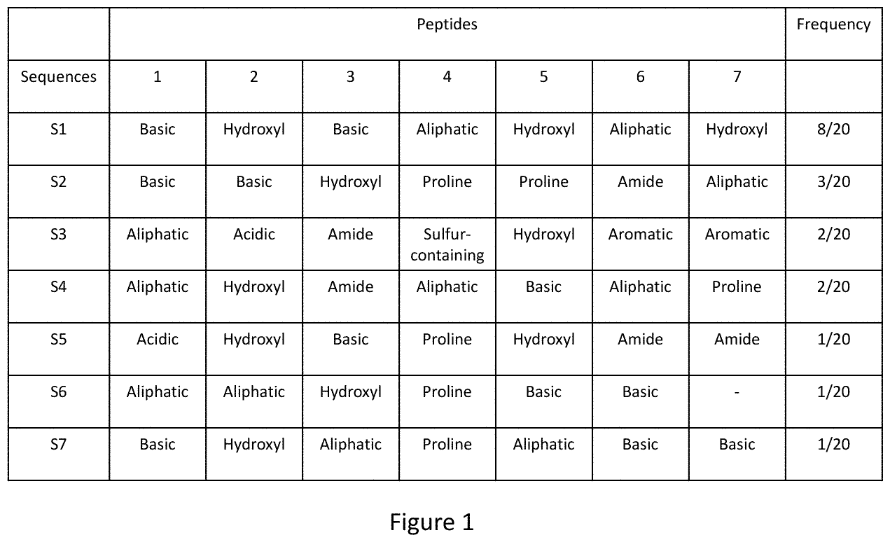

From screening and DNA sequencing, one unique peptide sequence, designated as S1 (FIG. 1), was present with a frequency of 40% from sequenced clones. Interestingly, two clones with the highest frequency, S1 and S2, were hydrophilic, as evidenced from the properties of the amino acid sequence (FIG. 1). The initial findings from these hits confirm prior work where hydrophilic polymers provided an inert surface minimizing mucin interactions and hydrophobic domains from drug delivery systems or viruses interact with hydrophobic pocket in mucin, resulting in mucin bundles and ultimately hindering particle transport (Wang et al., 2008, Angew Chem Int Ed Engl, 47: 9726-9729; Olmsted et al., 2001, Biophys J, 81: 1930-1937; Wang et al, 2001, PLoS One, 6: e21547).

Collectively, the results from this work provide design principles towards achieving the long-term goal of effectively delivering therapeutic and imaging agents through the mucosal barriers.

### Example 2: Using Combinatorial Biology to Develop Design Principles for Mucus-Penetrating Drug Delivery Systems

In mucosal-based diseases such as cystic fibrosis, the altered mucus microenvironment traps and protects pathogens resulting in chronic bacterial infections, while serving as a physical barrier to delivery of drugs. While recent studies using formulations of hydrophilic, net-neutral charge polymers are promising (Lai et al., 2007, Proc Natl Acad Sci USA, 104: 1482-1487), few have been tested and possess uniform physicochemical properties, which do not necessarily recapitulate the complexity seen in native mucus-substrate interactions (Li et al., 2013, Biophys J, 105: 1357-1365). Here phage display is used as “biological-based” screening tool to identify peptides with desired physicochemical properties for improved transport through mucus. Previously, phage display has been utilized as a technology to discover peptides with selective affinity for a broad spectrum of targets including antibodies, epitopes, small molecules and synthetic materials. The strategy described herein leverages the combinatorial diversity of these large, engineered libraries of random peptides (i.e. 10⁸-10⁹ different peptides) by using an unprecedented high-throughput approach to identify peptides with “stealth-like” physicochemical properties for enhanced mucosal transport.

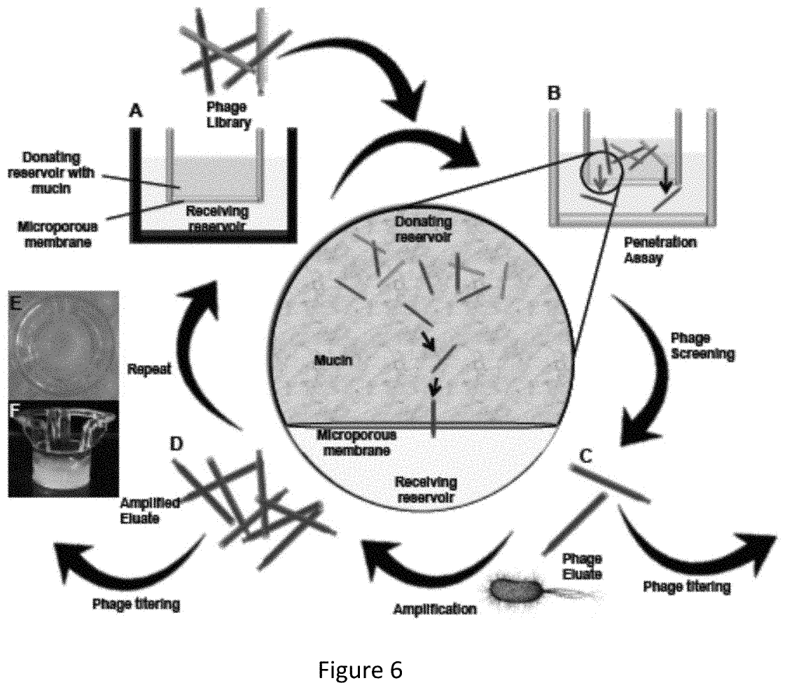

To identify phage-based peptides able to penetrate hyper-concentrated mucus, a screening assay was developed (FIG. 6). Phage libraries were incubated with 8% w/v mucin or complex mucin in a transwell chamber. Diffused phage were collected in the bottom reservoir at various time points and counted using standard plaque forming assay. Then, collected phage were amplified in bacteria to make more copies. These phage were added to mucin and screening was repeated to collapse the library to few phage-presenting peptides most able to penetrate the mucin. After three rounds of screening, individual phage sequences were isolated and identified by standard DNA sequencing. Sequences that demonstrated highest frequency were validated for improved transport compared to phage control (i.e. without peptide modifications). Diffusivity of screened clones were quantified using the transport assay and calculated using a previously developed method (Hu et al., 2010, Biotechnol Prog, 26: 1213-1221).

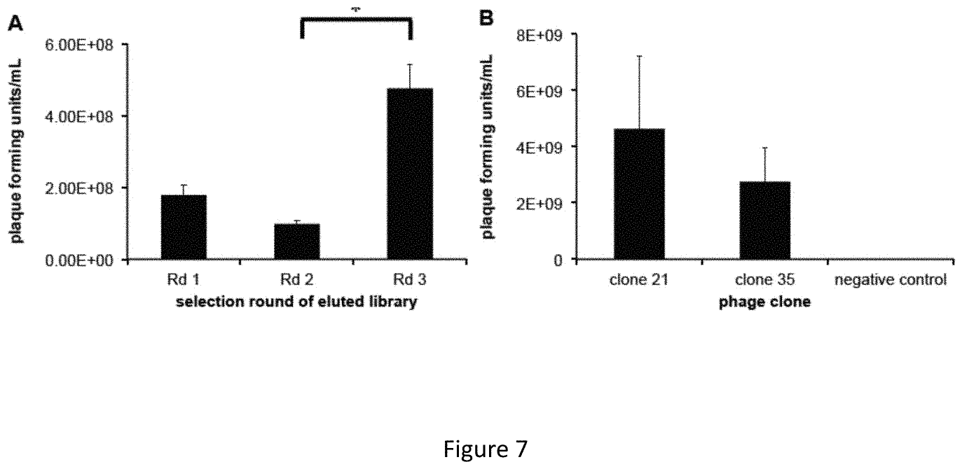

Experiments were conducted quantify and validate the phage transported through the hyperconcentrated mucin. FIG. 7A demonstrates the tittering results of phage eluate at 1 hour timepoint against a mucous layer. FIG. 7B is a comparison of tittering results of phage eluate at 1 hour timepoint between positive clones from round 3 and the wildtype negative controls.

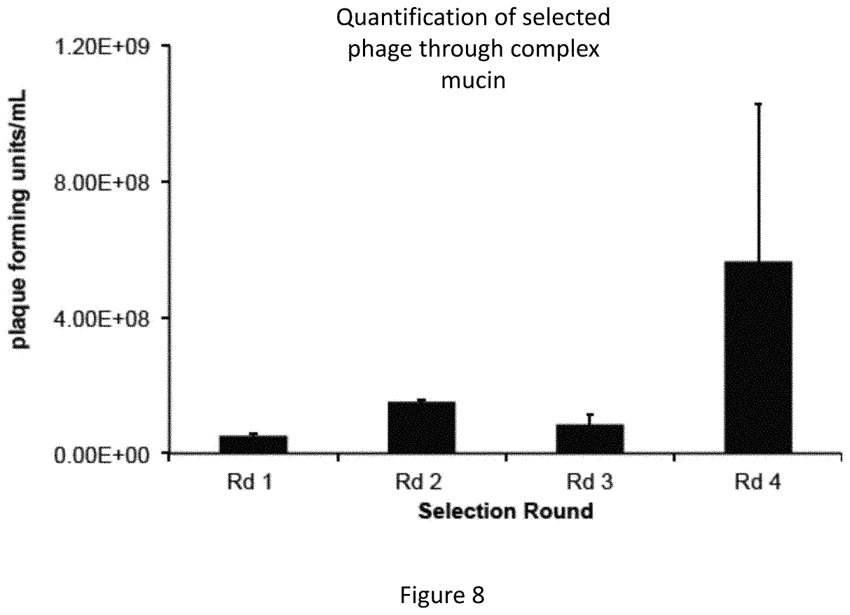

Experiments were then conducted to quantify selected phage through complex mucin. FIG. 8 depicts the tittering results of the phage eluate at 1 hour time point against a complex mucin formulation containing lipids, protein cell debris, and salts. An enrichment in the number of phages that are transported across the mucus layer can be seen markedly in round 4.

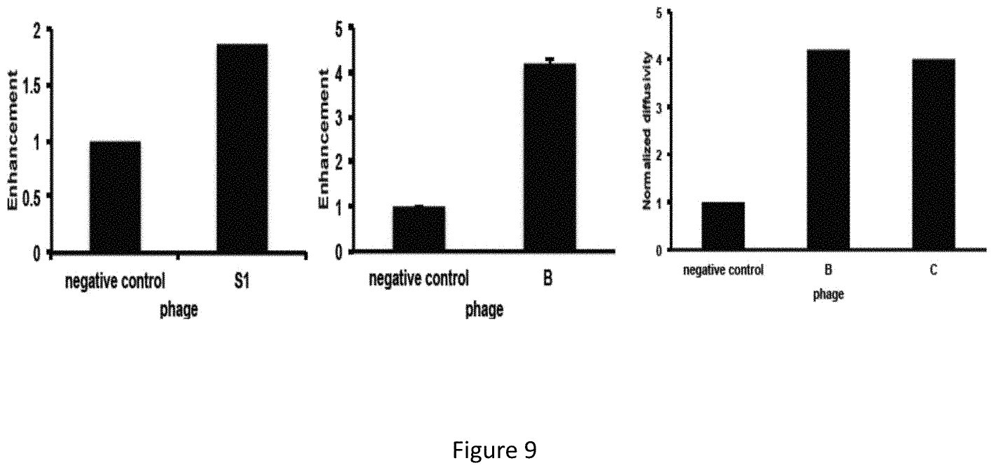

Experiments were then conducted to examine the diffusivity of selected phage. FIG. 9 depicts the enhanced diffusivity of selected mucin-penetrating M13 phage. The left panel of FIG. 9 depicts the diffusivity of selected phage S1 and a negative control in 8% mucin. The center and right panels of FIG. 9 depict the diffusivities of selected phage B and negative control in complex mucin.

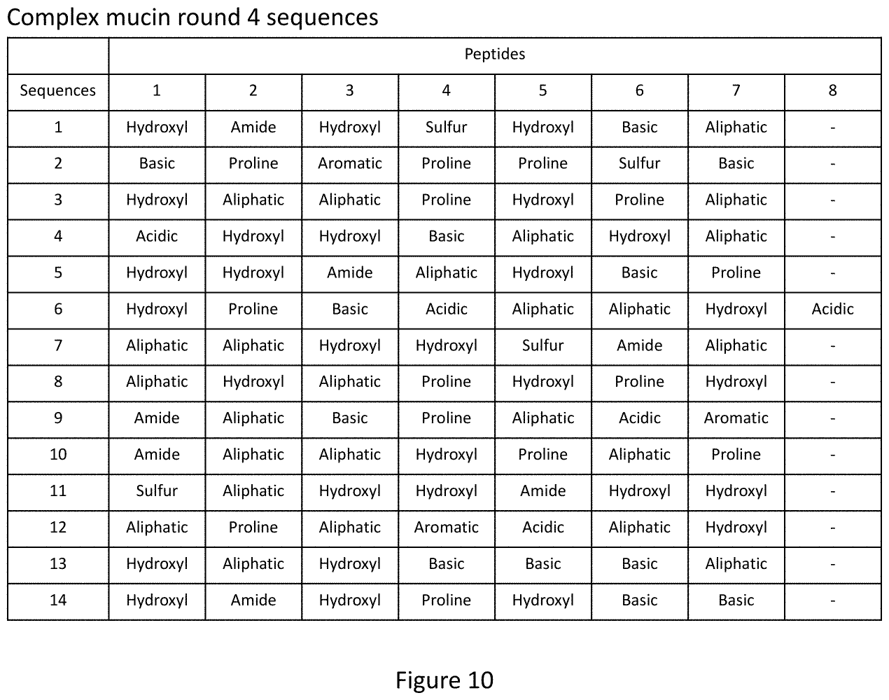

FIG. 10 is a table of 14 identified sequences from the 4th round eluates from complex mucin screens. Interestingly numerous identified sequences are hydrophilic (see color code) and this initial finding is consistent with prior work where hydrophilic polymers provided an inert surface minimizing mucin interactions (Lai et al., 2007, Proc Natl Acad Sci USA, 104: 1482-1487). 51 demonstrated improved diffusivity in mucin compared to the control phage, suggesting a role of the selected peptide to improve diffusivity through mucin.

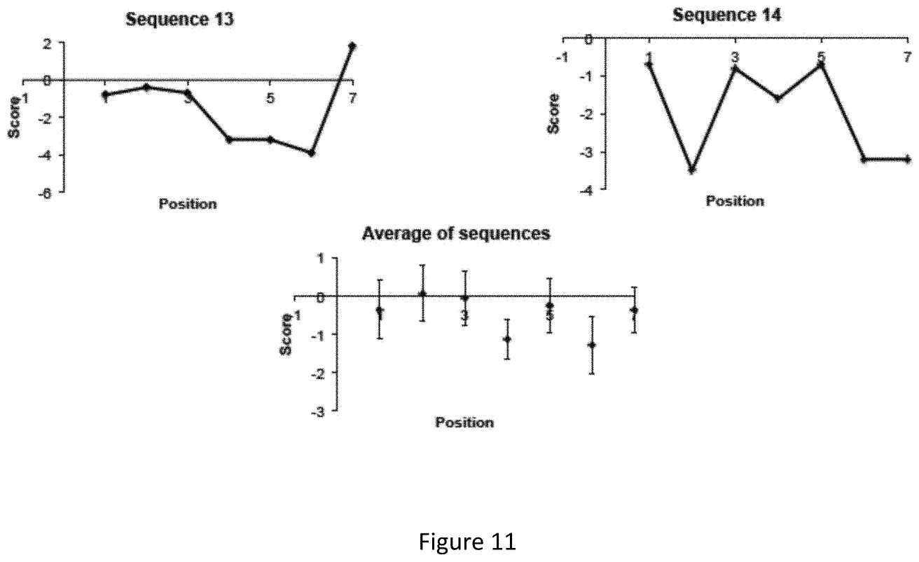

They hydrophilicity of the mucin-penetrating clones was examined. FIG. 11 depicts a Kyte-Doolittle hydropathy plot of sequences 13 and 14 from FIG. 10. X-axis is amino acid position and y-axis denotes hydropathy score assigned to amino acid. Negative score represents hydrophilic amino acids and positive scores represent hydrophobic amino acids. From the collected sequences, the average hydrophobicity score at each amino acid position is calculated. Adopted from Kyte and Doolittle, Journal of Molecular Biology. 157, 1982. As depicted in FIG. 11, the hydropathy score of the peptides are negative, demonstrating the hydrophilicity of the identified peptides.

As described herein, the screen has identified peptides which allow transport through mucin. Collectively, these results provide design principles towards achieving the long-term goal of effectively delivering drugs through the mucosal barriers.

### Example 3: Peptide Conjugates Identified Via Phage Display can Facilitate Transport of Molecules as Conjugates

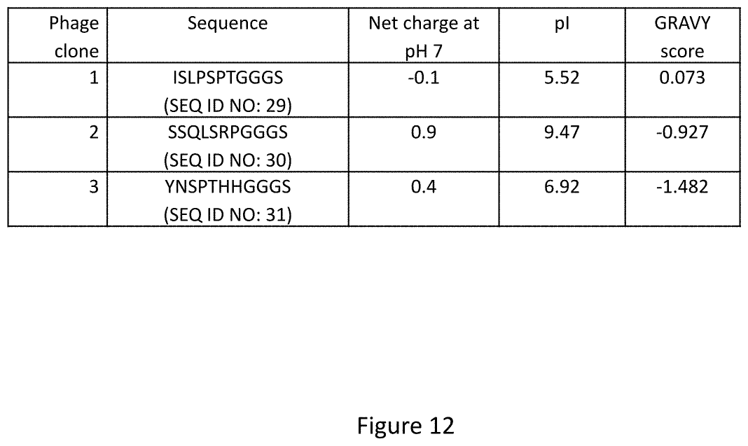

A library displaying linear random 7-mer peptides on the p3 coat of M13 bacteriophage were panned against complex mucin for four rounds to screen and identify peptide sequences that facilitate transport across the mucin barrier. Briefly, 1 μL of approximately 2*10¹⁰ plaque forming units of M13 Ph.D.™-7 Phage Display Peptide Library (New England Biolabs; diversity of 2*10⁹ (provided by manufacturer)) were mixed with complex mucin (0.73 g mucin type II (Sigma), 0.038 g lecithin, 0.39 g bovine serum albumin, 0.054 g NaCl, 240 μL 1 M HEPES, and diluted to 12 mL with sterile H₂O; stirred overnight at room temperature to mix) to a final volume of 500 μL. Next, the transwell assay was prepared. To a 12-well transwell (Corning), 1.5 mL PBS was added to the bottom of the transwell (i.e. receiving chamber). Next, 500 μL of the complex mucin premixed with the library was pipetted and dispensed into the top (donor) chamber, of the transwell. After 60 minutes, samples were collected from the receiving chamber, and 10 uL of the eluted, collected phage (total volume of −1.5 mL) were titered using standard plaque assay (using a 6-well agar plate as opposed to more standard 10-cm plate) to quantify the concentration of eluted phage. 1 mL of the remaining eluted phage were amplified and purified following manufacturers' recommendations to make more copies of eluted phage for a subsequent round of screening. This screening process was repeated for 3 more subsequent rounds—in total, four eluates were collected and the first three rounds of eluted phage were amplified for next round of screening. To confirm enrichment of phage screening (i.e. through screening against mucin, the process enriches for clones that do not adhere to the mucin and are able to penetrate through the reconstituted mucin barrier), the titers from each round of eluted phage were compared. As demonstrated herein, there is increased concentration (plaque forming units/mL) of eluted phage at round 4 compared to previous rounds (FIG. 12). The increased concentration of eluted phage is typical of enrichment of the pooled library of phage, as seen in traditional pannings.

After four rounds of screening against complex mucin, phage library-infecting bacteria were plated and overlaid with agar on LB-agar plates to obtain individual plaques (i.e. clones). Individual plaques were isolated and amplified in E. Coli cultures to grow more copies of isolated phage and thus more DNA of isolated phage. The DNA of the individual clones were purified using Qiagen QIAprep Spin Miniprep kit and sequenced by Sanger DNA sequencing.

From these clones, numerous sequences were identified. In particular, three clones encoding for peptide sequences displayed on the N-terminus of p3 of the library, were of particular interest:

1.
(SEQ ID NO: 17)
ISLPSPT
2.
(SEQ ID NO: 14)
SSQLSRP
3.
(SEQ ID NO: 19)
YNSPTHEI

These sequences were chosen due to the presence of hydrophilic residues serine (S) and threonine (T). Previous literature suggests that hydrophilic surface chemistries enhance transport of particles/drug carriers through mucin barrier. The physicochemical properties of these sequences (the flexible linker GGGS is added because that is engineered into the p3 library for N-terminal display of peptides) is provided in FIG. 12.

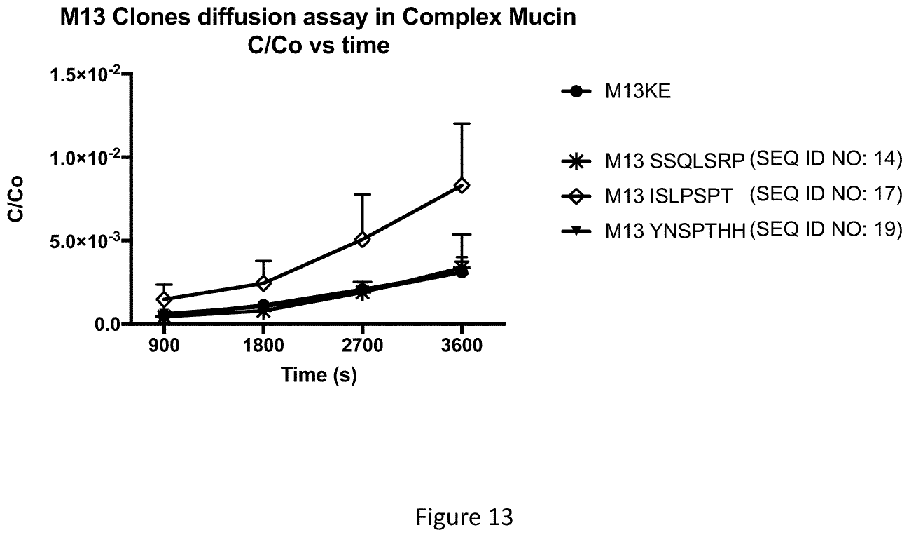

To quantify their role in facilitating diffusion across the mucin barrier, individual phage clones were incubated with mucin, and phage were collected at 15, 30, 45 and 60 minutes. Each timepoint of the collected phage (of each clone) were titered and compared to the initial concentration. From the slope, the diffusion coefficient of the phage clones can be quantified. A plot of the ratio of concentrations of clones diffusing through mucin barrier at a given time to the initial phage concentration (concentration at time 0 sec) was created. The bulk diffusion of these clones is compared to M13 phage without library/peptide insert (denoted as M13KE) (FIG. 13).

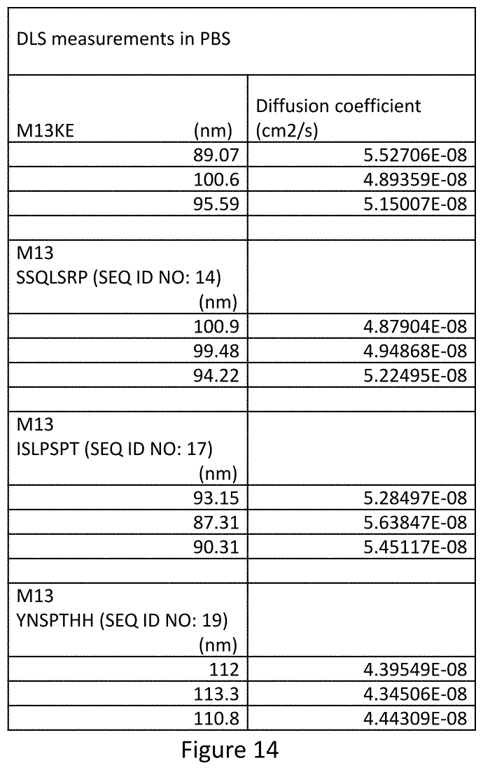

It was desired to compare the diffusive behavior of the clones in mucin to their diffusion in an unhindered medium, i.e. PBS. Since the diffusion in PBS can be rapid, it is not necessarily amenable to the bulk diffusion transwell assay, and the diffusion of the phage clones was determined by dynamic light scattering (DLS). From dynamic light scattering, one can calculate the diffusion coefficient of the clones in a non-viscous, unhindered barrier. A table of the diffusivity of phage clones and M13KE in PBS is provided in FIG. 14. From the data, the diffusivities of the phage clones and M13KE in PBS are similar.

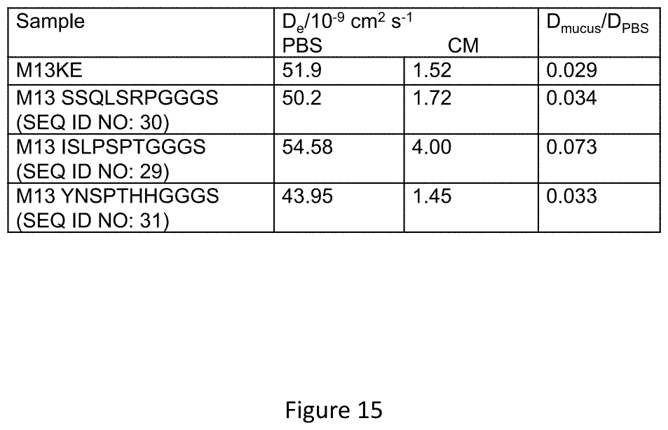

When the effective diffusivity of clones in complex mucin (CM) is compared to the effective diffusivity of clones in PBS, the effect of the peptide on the phage body to facilitate transport is demonstrated. A table comparing phage diffusivities in PBS, CM, and the ratio of diffusivity in CM to PBS is provided in FIG. 15. There is increased phage diffusivity with ISLPSPT (SEQ ID NO: 17).

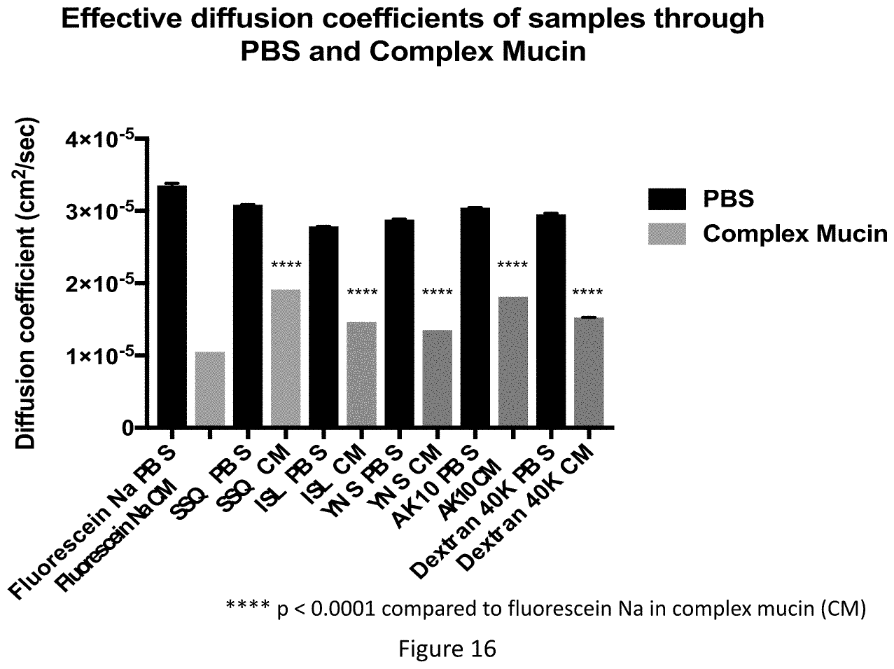

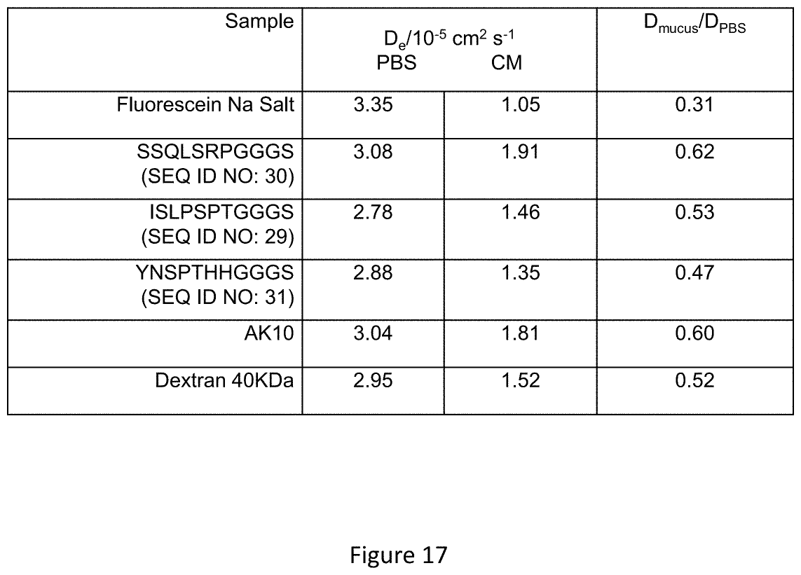

From the DLS measurements, the peptide ISLPSPT (SEQ ID NO: 17) on phage influences transport. However, previous data showed diffusivity of phage. To validate identified peptides out of the structural context of phage demonstrate improved diffusivity and demonstrate that peptides improve diffusivity of molecular conjugates (e.g. peptide-drug conjugates to improve penetration of drug through mucin barrier), fluorescein isothiocyanate (FITC) dyes were conjugated with synthetic peptides and their diffusivity through the mucin barrier was compared with fluorescein salt without any conjugated peptide (salt is a water soluble form of fluorescein and comparable dye to FITC). Fluorescently labeled peptides and fluorescein were added to a layer of complex mucin (CM) in a transwell, and samples that penetrated through CM or PBS at 60 minutes were collected from the receiving chamber and the diffusivities were calculated and compared to fluorescein. All peptide conjugates (i.e. peptides conjugated to FITC) demonstrated significantly improved effective diffusivities (De) compared with fluorescein salt in CM (FIG. 16; all unpaired t-tests performed comparing the peptides versus fluorescein salt in complex mucin had a p<0.0001) and had better ratios of transport through CM relative to transport through PBS (also see FIG. 17; FIG. 16 is a graphical representation of the values in FIG. 17). AK10 and Dextran 40 kDa served as controls). These findings suggest that peptide conjugates identified via phage display can facilitate transport of molecules as conjugates.

### Example 4: Use of T7 Phage Libraries to Identify Mucous-Penetrating Peptides

The initial studies employed M13 phage display peptide libraries on the p3 coat proteins to identify peptides that can facilitate transport. There are approximately 5 copies of peptide on the p3 coat, and the bulk of the M13 is wild-type p8 coat protein, and may be involved in interactions with the mucin barrier. Additionally, phage clones were identified by Sanger sequencing; the sample space is limited due to the time to isolate and identify individual clones. To address these challenges, T7 phage libraries (random 7-mer peptides constrained by flanking cysteines) were genetically engineered for display on the C-terminus of gp10A protein of icosahedral lytic T7 bacteriophage (T7Select 415-1, Millipore). Engineering onto the C-terminus of gp10A allows for 415 copies of peptide displayed completely over the coat of the virus; this increases the surface area of the peptides to have more multivalent interactions (or lack thereof) with the mucin barrier. Also, to increase the number of identified clones and more accurately search for enriched clones penetrating through the mucin barrier (e.g. minimize false positives), next-generation sequencing (NGS) with bioinformatics analysis was utilized.

T7 phage libraries were incubated with complex mucin in a transwell, and samples were collected at 15, 30, 45, and 60 min in the receiving chamber (similar to M13 phage library screening). All collected timepoints were titered; samples at 60 minutes were further amplified following manufacturers' protocol (Millipore) for subsequent rounds of screening. Panning against mucin was continued for a total of three rounds. Herein, DNA from the pooled libraries from each round of panning, timepoint and replicate (panning was done in duplicate) are considered a separate experiment and the pooled library DNA was isolated (as opposed to individual isolation for Sanger sequencing). Next, for each experiment, the DNA was PCR amplified to attach and label barcodes to identify and separate the sequences during next generation sequencing (NGS) analysis. Samples were run on Illumina MiSeq. R and Python scripts were developed to translate the DNA sequences corresponding to peptides (from library panning), and their frequencies were calculated for each experiment (i.e. round, timepoint and replicate).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising at least one selected from the group consisting of:
a.) one or more mucus-penetrating peptides; and
b.) one or more isolated nucleic acid molecules encoding one or more mucus-penetrating peptides.

2. The composition of claim 1, wherein the one or more mucus-penetrating peptides comprises a peptide comprising an amino acid sequence selected from the group consisting of: an amino acid sequence selected from SEQ ID NOs: 1-28, an amino acid sequence having at least 70% homology to any one of SEQ ID NOs: 1-28, and a fragment of an amino acid sequence selected from SEQ ID NOs: 1-28.

3. The composition of claim 1, wherein the one or more mucus-penetrating peptides comprises a peptide comprising an amino acid sequence selected from SEQ ID NOs: 1-28.

4. The composition of claim 1, wherein the composition further comprises at least one agent selected from the group consisting of: a therapeutic agent, prophylactic agent, diagnostic agent, imaging agent, contrast agent, microparticle, and nanoparticle.

5. The composition of claim 4, wherein the agent is at least one selected from the group consisting of a peptide, nucleic acid molecule, small molecule drug, organic compound, and inorganic compound.

6. The composition of claim 4, wherein the composition comprises a fusion construct comprising one or more mucus-penetrating peptides conjugated to the at least one agent.

7. (canceled)

8. The composition of claim 1, wherein the one or more isolated nucleic acid molecules encodes a mucus-penetrating peptide comprising an amino acid sequence selected from the group consisting of: an amino acid sequence selected from SEQ ID NOs: 1-28, an amino acid sequence having at least 70% homology to any one of SEQ ID NOs: 1-28, and a fragment of an amino acid sequence selected from SEQ ID NOs: 1-28.

9. A method of delivering an agent across a mucosal barrier comprising administering to the mucosal barrier a composition comprising the agent and one or more mucus-penetrating peptides.

10. The method of claim 9, wherein the one or more mucus-penetrating peptides comprises a peptide comprising an amino acid sequence selected from the group consisting of: an amino acid sequence selected from SEQ ID NOs: 1-28, an amino acid sequence having at least 70% homology to any one of SEQ ID NOs: 1-28, and a fragment of an amino acid sequence selected from SEQ ID NOs: 1-28.

11. The method of claim 9, wherein the agent is at least one selected from the group consisting of a therapeutic agent, prophylactic agent, diagnostic agent, imaging agent, contrast agent, microparticle, and nanoparticle.

12. The method of claim 9, wherein composition comprises a fusion construct comprising the one or more mucus-penetrating peptides conjugated to the agent.

13. The method of claim 9, wherein the composition comprises a therapeutic or prophylactic agent and one or more mucus-penetrating peptides, and wherein the method comprises administering the composition to a subject.

14. The method of claim 13, wherein the one or more mucus-penetrating peptides comprises a peptide comprising an amino acid sequence selected from the group consisting of: an amino acid sequence selected from SEQ ID NOs: 1-28, an amino acid sequence having at least 70% homology to any one of SEQ NOs: 1-28, and a fragment of an amino acid sequence selected from SEQ ID NOs: 1-28.

15. The method of claim 13, wherein composition comprises a fusion construct comprising the one or more mucus-penetrating peptides conjugated to the therapeutic or prophylactic agent.

16. A method of screening for a compound capable of penetrating a mucosal barrier comprising:
providing a container comprising a first chamber, a second chamber, and a permeable membrane separating the first chamber and second chamber, wherein the first chamber comprises mucus or mucus-like substance;
administering one or more test compounds to the first chamber; and
collecting the contents of the second chamber at a time point following the administration of the one or more test compounds.

17. The method of claim 16, wherein the method further comprises one or more rounds of re-administering the collected contents of the second chamber into the first chamber and collecting the contents of the second chamber.

18. The method of claim 16, wherein the method comprises a phage library-based assay, comprising administering a plurality of peptide-expressing phage to the first chamber, and collecting the phage in the second chamber.
